(19) United States
(12) Patent Application Publication
(10) Pub. No.: US 2021/0052478 A1
(43) Pub. Date: Feb. 25, 2021

(54) PERSONAL CARE COMPOSITIONS

(71) Applicant: ALTUS LABS, LLC, Denver, CO (US)

(72) Inventors: Helena YARDLEY, Denver, CO (US); Michael TAGEN, Denver, CO (US); Jonathan FERNANDEZ, Denver, CO (US); Robert DELLAVALLE, Denver, CO (US); Irwin PALEFSKY, Fairfield, NJ (US); Nicolle GONZALES, Denver, CO (US)

(21) Appl. No.: 16/960,188

(22) PCT Filed: Jan. 7, 2019
(86) PCT No.: PCT/US2019/012488

§ 371 Date: Jul. 6, 2020

Related U.S. Application Data
(60) Provisional application No. 62/698,748, filed on Jul. 16, 2018.
(60) Provisional application No. 62/652,901, filed on Apr. 5, 2018.
(60) Provisional application No. 62/614,317, filed on Jan. 5, 2018.

(51) Int. Cl. A61K 8/49; A61K 8/34; A61K 8/9794; A61K 8/92; A61K 8/42

(52) U.S. Cl. CPC A61K 8/498; A61K 8/347; A61K 2800/34; A61K 8/922; A61K 8/42; A61K 8/9794

(57) ABSTRACT

Described herein are personal care compositions and methods of making and using same. A personal care composition comprising: a cannabinoid; a PPAR-a agonist; a terpene; and a cosmetically acceptable carrier.

### BACKGROUND

While numerous agents are known to provide benefits for chronic or acute pain and diseases, disorders and conditions of the skin, many of these agents are incompatible. As such, formulators are not only faced with the challenge of combining these ingredients during manufacture, but they must also ensure that the end product has a consumer acceptable texture/skin-feel; and is physically stable for the duration of its shelf life.

Many of these agents have supporting evidence as a method of treating, preventing or ameliorating a symptom associated with a disease, disorder, or condition relating to both chronic or acute pain, through topical or transdermal preparations. However, targeted delivery of the active ingredients to the physical location, and depth at that location, along with the aforementioned texture/skin-feel can create complex technological formulation challenges.

Additional formulation challenges are presented by concerns related to certain ingredients (e.g. cyclic silicones) or derivative products formed in situ (e.g. n-nitroso compounds), that are potential carcinogens.

As such, there remains a need for a combination of safe ingredients that provide the desired end-benefit, while also being easy to manufacture, consumer acceptable, and physically stable. Certain embodiments of the present invention are directed to meeting these needs.

### SUMMARY

Some embodiments of the present invention provide a personal care composition comprising: a cannabinoid; a PPAR-α agonist; and a cosmetically acceptable carrier. In some embodiments, the cannabinoid is a phytocannabinoid. In other embodiments, the cannabinoid is a synthetic cannabinoid.

Other embodiments of the present invention provide a personal care composition comprising: from about 0.1 wt. % to about 10 wt. %, of a cannabinoid; from about 0.1 wt. % to about 5 wt. % of a PPAR-α agonist; and from about 80 wt. % to about 95 wt. % of a cosmetically acceptable carrier.

Certain embodiments provide a personal care composition comprising: a cannabinoid; a terpene; and a cosmetically acceptable carrier.

Yet other embodiments provide a personal care composition comprising: a first hydrophobic material comprising a semi-solid mixture of hydrocarbons; a second hydrophobic material comprising a complex comprising a paraffin wax and a microcrystalline wax; a cannabinoid; a terpene; and a cosmetically acceptable carrier.

In some embodiments, the compositions described herein further comprise a skin protectant system. In other embodiments, the compositions described herein further comprise a penetration enhancing system.

Some embodiments of the present invention provide personal care compositions that are substantially free of cyclic silicones. Other embodiments of the present invention provide personal care compositions that are free of cyclic silicones.

Further embodiments of the present invention provide methods of treating, preventing or ameliorating a symptom associated with a disease, disorder or condition of the skin, comprising applying any one of the compositions described herein to the skin of a subject in need thereof. In some embodiments, the personal care compositions comprise a combination of ingredients that are effective regardless of the base in which they are incorporated.

### DETAILED DESCRIPTION

As used herein, the term “personal care composition” is intended to include, but not be limited to, products such as cleansers, hair care products (e.g. shampoos, mousses and conditioners) lotions, creams, salves and ointments. These products may be delivered from wipes (e.g. nonwoven substrates), containers, tubes, sachets, patches, pumps or sticks, and the like.

In some embodiments, the compositions described herein are suitable for transdermal administration. In certain embodiments, the compositions described herein are adapted for administration in and/or around the abdomen, back, chest, legs, arms, scalp or other suitable skin surface and may include formulations in which the cannabinoid(s) are administered in patches or through a matrix or mesh, and the like.

Still further embodiments provide transdermal methods of delivery whereby cannabinoids can be delivered and be made systemically available to a mammal in a therapeutically effective amount. In addition to the benefits of systemically administered cannabinoids, there are also localized benefits from topical administration. For example, topically administered cannabinoids are useful to alleviate pain and other conditions originating near the surface of the skin.

As used herein, “cannabinoid” is intended to encompass both acidic and neutral forms, and may refer to individual cannabinoids or combinations thereof.

Some embodiments of the present invention provide a personal care composition comprising: a cannabinoid; a PPAR-α agonist; and a cosmetically acceptable carrier. In some embodiments, the cannabinoid is a phytocannabinoid. In other embodiments, the cannabinoid is a synthetic cannabinoid. In some embodiments, the cannabinoid is in a neutral form. In other embodiments, the cannabinoid is in an acidic form.

Unless otherwise indicated, all percentages and amounts in the present application are defined as weight percentages (w/w % or wt. %).

In some embodiments, the phytocannabinoid is selected from: tetrahydrocannabinol; tetrahydrocannabinolic acid; cannabidiol; cannabidiolic acid; cannabinol; cannabigerol; cannabichromene; cannabicyclol; cannabivarin; tetrahydroc annabivarin; cannabidivarin; cannabichromevarin; cannabigerovarin; cannabigerol monomethyl ether; cannabielsoin; cannabicitran; and a combination of two or more thereof. In other embodiments, the phytocannabinoid is selected from tetrahydrocannabinol; cannabidiol; and a combination thereof. In some embodiments, the phytocannabinoid is tetrahydrocannabinol. Still further embodiments provide personal care compositions wherein the phytocannabinoid is cannabidiol. Yet other embodiments provide personal care compositions comprising a combination of tetrahydrocannabinol and cannabidiol. In some embodiments, the tetrahydrocannabinol is delta-9-tetrahydrocannabinol. In some embodiments, the active ingredient in the personal care compositions described herein is a prodrug, derivative, or metabolite of any one of the cannabinoids described herein.

Various ratios of the above-described cannabinoids can be used in the compositions described herein. The ratios can be adjusted based on pharmacological effects required. For example, particular cannabinoids can be enriched and/or purified from a cannabis extract via techniques such as fractional distillation or adjusting the harvesting technique of the plants. Ratios of enriched/purified cannabinoids for the cannabinoid products of the invention can be adjusted, such as, for example, 1:1 w/w CBD:THC. Ratios include 0.1:1, 0.2:1, 0.3:1, 0.4:1, 0.5:1, 0.6:1, 0.7:1, 0.8:1, 0.9:1, 1:1, 1:1.2, 1:1.5, 1:1.3, 1:1.5, 1:1.7, 1:2, 1:3, 1:4, 1:5, 1:6, 1:7, 1:8 or 1:10 (all ratios given are w/w).

In some embodiments individual doses of the compositions of the present invention contain from about 0.1 to about 100 milligrams (mg) of a cannabinoid, from about 0.5 to about 50 mg, from about 1 to about 40 mg, from about 2 to about 20 mg, from about 5 mg to about 15 mg, or about 0.1 mg, 0.5 mg, 1 mg, 2 mg, 3 mg, 4 mg, 5 mg, 6 mg, 8 mg, 10 mg, 12 mg, 14 mg, 16 mg, 18 mg, 20 mg, 30 mg, 40 mg, 50 mg, 60 mg, 80 mg or more per dose.

Some embodiments provide personal care compositions further comprising a terpene. For example, a personal care composition comprising a cannabinoid, a PPAR-α agonist and a terpene. In some embodiments, the terpene is selected from: from 7,8-dihydroionone, Acetanisole, Acetyl Cedrene, Anethole, Anisole, Benzaldehyde, Bergamotene (α-cis-Bergamotene) (α-trans-Bergamotene), Bisabolol (α-Bisabolol), Borneol, Butanoic/Butyric Acid, Cadinene (α-Cadinene) (γ-Cadinene), Cafestol, Caffeic acid, Camphene, Camphor, Capsaicin, Carene (Δ-3-Carene), Carotene, Carvacrol, Carvone, Dextro-Carvone, Laevo-Carvone, Caryophyllene (β-Caryophyllene), Caryophyllene oxide, Castoreum Absolute, Cedrene (α-Cedrene) (β-Cedrene), Cedrene Epoxide (α-Cedrene Epoxide), Cedrol, Cembrene, Chlorogenic Acid, Cinnamaldehyde (α-amyl-Cinnamaldehyde) (α-hexyl-Cinnamaldehyde), Cinnamic Acid, Cinnamyl Alcohol, Citronellal, Citronellol, Cryptone, Curcumene (α-Curcumene) (γ-Curcumene), Decanal, Dehydrovomifoliol, Diallyl Disulfide, Dihydroactinidiolide, Dimethyl Disulfide, Eicosane/Icosane, Elemene (β-Elemene), Estragole, Ethyl acetate, Ethyl Cinnamate, Ethyl maltol, Eucalyptol/1,8-Cineole, Eudesmol (α-Eudesmol) (μ-Eudesmol) (γ-Eudesmol), Eugenol, Euphol, Farnesene, Farnesol, Fenchol (β-Fenchol), Fenchone, Geraniol, Geranyl acetate, Germacrenes, Germacrene B, Guaia-1(10), 11-diene, Guaiacol, Guaiene (α-Guaiene), Gurjunene (α-Gurjunene), Herniarin, Hexanaldehyde, Hexanoic Acid, Humulene (α-Humulene) (β-Humulene), Ionol (3-oxo-α-ionol) (β-Ionol), Ionone (α-Ionone) (β-Ionone), Ipsdienol, Isoamyl acetate, Isoamyl Alcohol, Isoamyl Formate, Isoborneol, Isomyrcenol, Isopulegol, Isovaleric Acid, Isoprene, Kahweol, Lavandulol, Limonene, γ-Linolenic Acid, Linalool, Longifolene, α-Longipinene, Lycopene, Menthol, Methyl butyrate, 3-Mercapto-2-Methylpentanal, Mercaptan/Thiols, β-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (β-Myrcene), γ-Muurolene, Nepetalactone, Nerol, Nerolidol, Neryl acetate, Nonanaldehyde, Nonanoic Acid, Ocimene, Octanal, Octanoic Acid, P-cymene, Pentyl butyrate, Phellandrene, Phenylacetaldehyde, Phenylethanethiol, Phenylacetic Acid, Phytol, Pinene, β-Pinene, Propanethiol, Pristimerin, Pulegone, Retinol, Rutin, Sabinene, Sabinene Hydrate, cis-Sabinene Hydrate, trans-Sabinene Hydrate, Safranal, α-Selinene, α-Sinensal, β-Sinensal, β-Sitosterol, Squalene, Taxadiene, licorice root extract or a derivative thereof (e.g. glycyrrhizic acid or glycyrrhetinic acid), Terpin hydrate, Terpineol, Terpine-4-ol, α-Terpinene, γ-Terpinene, Terpinolene, Thiophenol, Thujone, Thymol, α-Tocopherol, Tonka Undecanone, Undecanal, Valeraldehyde/Pentanal, Verdoxan, α-Ylangene, Umbelliferone, Vanillin; and a combination of two or more thereof. In some embodiments, the terpene comprises bisabolol (α-bisabolol). In some embodiments, the terpene comprises licorice root extract, or a derivative thereof (e.g. glycyrrhizic acid or glycyrrhetinic acid). Other embodiments comprise a salt of a licorice root extract or a derivative thereof.

In some embodiments, a terpene is included in the composition generally between about (101% and about 20% of the composition, between about 0.02% and about 17.5%, between about 0.03% to about 15%, between about 0.04% to about 12.5%, between about 0.05% and about 10%, between about 0.075% and about 7.5%, between about 0.1% and about 5%, between about 0.15% and about 4%, between about 0.2% and about 3%, between about 0.25% and about 2%, between about 0.3% and about 1.5%, or between about 0.4% and 1%, or about 0.5%. Alternatively, the terpene/terpenoid amount can be about 0.01%, about 0.05%, about 0.1%, about 0.15%, about 0.2%, about 0.25%, about 0.3%, about 0.35%, about 0.4%, about 0.45%, about 0.5%, about 0.55%, about 0.6%, about 0.7%, about 0.8%, or about 1% or more.

In some embodiments, the term “terpene” is used interchangeably with the terms “terpenoid” and “isoprenoid”.

Other embodiments provide personal care compositions further comprising a flavonoid. In some embodiments, the flavonoid is selected from: a phenolic acid, a stilbenoid, a dihydroflavonol, an anthocyanin, an anthocyanidin, a polyphenol, a flavone, a flavan-3-ol, a flavan-4-ol, a flavan-3,4-diol flavonol, an antioxidant, a homoisoflavonoid, a phenylpropanoid, a steroid glycoside, a bioflavonoid, an isoflavonoid, a neoflavonoid, and a combination of two or more thereof. Still further embodiments provide compositions wherein the flavonoid is selected from: quercetin; rutin; quercitrin; catechin; gallocatechin; catechin 3-gallate; gallocatechin 3-gallate; epicatechin; epigallocatechin; epicatechin 3-gallate; epigallocatechin 3-gallate; theaflavin-3-gallate; theaflavin-3′-gallate; theaflavin-3,3′-digallate; kaempferol; myricetin; fisetin; galangin; isorhamnetin; pachypodol; rhamnazin; a pyranoflavonol; furanoflavonol; luteolin; resveratrol; apigenin; tangeritin; hesperetin; naringenin; eriodictyol; homoeriodictyol; and a combination of two or more thereof. Some embodiments provide personal care compositions wherein the flavonoid is selected from quercetin; luteolin; resveratrol; and a combination of two or more thereof.

Further embodiments provide any one of the personal care compositions described herein, further comprising menthol. In some embodiments, the personal care composition comprises from about 0.1 wt. % to about 10 wt. % menthol. In other embodiments, the personal care composition comprises from about 0.5 wt. % to about 5 wt. % menthol. Still further embodiments provide personal care compositions comprising from about 1 wt. % to about 3 wt. % menthol. While other embodiments provide personal care compositions comprising about 2 wt. % menthol.

In some embodiments, the personal care compositions comprise a penetration enhancer. As used herein, the term “penetration enhancer” includes any agent, or combination thereof, that improves the transport of a molecule (e.g. an active ingredient) into or through the skin. In some embodiments, the penetration enhancer(s) can be included in the composition is between about 0.1% and about 20% of the composition, between about 0.15% and about 15%, between about 0.2% and about 10%, between about 0.4% and about 8%, between about 0.6% and about 6%, between about 0.8% and about 4%, between about 0.9% and about 2%, or about 1%. Alternatively, the penetration enhancer amount can be about 0.1%, about 0.2%, about 0.4%, about 0.5%, about 0.6%, about 0.8%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5% or about 10%, by weight, of the composition.

In some embodiments, the penetration enhancer is selected from glyceryl oleate, ethanol, dimethyl sulfoxide, N-lauroyl sarcosine, ethoxydiglycol, dimethyl isosorbide, sodium lauryl sulfoacetate, isopropyl myristate, methyl laurate, propylene glycol, oleic acid, sodium octyl sulfate, eucalyptol, and any combination of two or more thereof.

In some embodiments, the present invention provides personal care compositions comprising a penetration enhancing system. In some embodiments, the penetration enhancing system comprises ethoxydiglycol. In other embodiments, the penetration enhancing system comprises from about 0.1 wt. % to about 10 wt. % ethoxydiglycol. In some embodiments, the penetration enhancing system comprises from about 0.5 wt. % to about 10 wt. % ethoxydiglycol. In some embodiments, the penetration enhancing system comprises from about 1 wt. % to about 10 wt. % ethoxydiglycol. In some embodiments, the penetration enhancing system comprises from about 1 wt. % to about 9 wt. % ethoxydiglycol. In some embodiments, the penetration enhancing system comprises from about 1 wt. % to about 8 wt. % ethoxydiglycol. In further embodiments, the penetration enhancing system comprises from about 1 wt. % to about 7.5 wt. % ethoxydiglycol. Still other embodiments provide personal care compositions wherein the penetration enhancing system comprises from about 2 wt. % to about 7 wt. % ethoxydiglycol. Yet other embodiments provide personal care compositions wherein the penetration enhancing system comprises from about 2.5 wt. % to about 7 wt. % ethoxydiglycol. While other embodiments provide personal care compositions wherein the penetration enhancing system comprises from about 3 wt. % to about 7 wt. % ethoxydiglycol. In some embodiments, the penetration enhancing system comprises from about 4 wt. % to about 6 wt. % ethoxydiglycol. In some embodiments, the penetration enhancing system comprises about 5 wt. % ethoxydiglycol.

In some embodiments, the penetration enhancing system comprises dimethyl isosorbide alone or in combination with any other penetration enhancer described herein. In some embodiments, the penetration enhancing system comprises from about 0.05 wt. % to about 7 wt. % dimethyl isosorbide. In other embodiments, the penetration enhancing system comprises from about 0.1 wt. % to about 6 wt. % dimethyl isosorbide. In other embodiments, the penetration enhancing system comprises from about 0.1 wt. % to about 5 wt. % dimethyl isosorbide. In other embodiments, the penetration enhancing system comprises from about 0.5 wt. % to about 4 wt. % dimethyl isosorbide. Still further embodiments provide personal care compositions wherein the penetration enhancing system comprises from about 1 wt. % to about 3 wt. % dimethyl isosorbide. Yet other embodiments provide personal care compositions wherein the penetration enhancing system comprises about 2 wt. % dimethyl isosorbide.

In some embodiments, penetration enhancing system comprises a first penetration enhancer and a second penetration enhancer. In some embodiments, the weight ratio of the first penetration enhancer to the second penetration enhancer is in the range of from about 1:10 to about 10:1. In some embodiments, the weight ratio of the first penetration enhancer to the second penetration enhancer is in the range of from about 1:9 to about 9:1. In some embodiments, the weight ratio of the first penetration enhancer to the second penetration enhancer is in the range of from about 1:8 to about 8:1. In further embodiments, the weight ratio of the first penetration enhancer to the second penetration enhancer is in the range of from about 1:7 to about 7:1. Still further embodiments provide compositions wherein the weight ratio of the first penetration enhancer to the second penetration enhancer is in the range of from about 1:6 to about 6:1. In some embodiments, the weight ratio of the first penetration enhancer to the second penetration enhancer is in the range of from about 1:5 to about 5:1. In some embodiments, the weight ratio of the first penetration enhancer to the second penetration enhancer is in the range of from about 1:4 to about 4:1. In still further embodiments, the weight ratio of the first penetration enhancer to the second penetration enhancer is in the range of from about 1:3 to about 3:1. Other embodiments provide compositions wherein the weight ratio of the first penetration enhancer to the second penetration enhancer is in the range of from about 1:2 to about 2:1. In certain embodiments, the weight ratio of the first penetration enhancer to the second penetration enhancer is in the range of from about 2:5 to about 5:2. In certain embodiments, the weight ratio of the first penetration enhancer to the second penetration enhancer is in the range of from about 3:4 to about 4:3. In certain embodiments, the weight ratio of the first penetration enhancer to the second penetration enhancer is about 1:1.

In some embodiments, the terms “penetration enhancing system” and “penetration enhancement system” are used interchangeably.

In some embodiments, the weight percent used in the composition will also be dependent upon the weight percent of the desired cannabinoid in the cannabis preparation and will be adjusted so that the ratio of the penetration enhancer to amount of desired cannabinoid is constant.

In some embodiments, the total concentration of the first penetration enhancer to the second penetration enhancer is in the range of from about 0.5 wt. % to about to about 15 wt. %, of the composition. In other embodiments, the total concentration of the first penetration enhancer to the second penetration enhancer is in the range of from about 0.5 wt. % to about 10 wt. %, of the composition. Still further embodiments provide compositions wherein the total concentration of the first penetration enhancer to the second penetration enhancer is in the range of from about 0.5 wt. % to about 7.5 wt. %, of the composition. In certain embodiments, the total concentration of the first penetration enhancer to the second penetration enhancer is in the range of from about 0.5 wt. % to about 5 wt. %, of the composition. Yet other embodiments provide compositions wherein, the total concentration of first penetration enhancer to the second penetration enhancer is in the range of from about 2 wt. % to about 10 wt. %, of the composition.

Other embodiments provide personal care compositions wherein the PPAR-α agonist comprises a fatty acid amide. In some embodiments, the fatty acid amide is an acyl-ethanolamide. In some embodiments, the fatty acid amide comprises palmitoylethanolamide (PEA). GMP grade PEA (OptiPEA®) is available from Innexus Nutraceuticals. In some embodiments, the fatty acid amide is a gpr55 agonist. In some embodiments, the fatty acid amide is a positive allosteric modulator of a cannabinoid receptor. In some embodiments, the fatty acid amide helps to mimic the skin's natural barrier.

In some embodiments, the personal care compositions of the present invention include a cosmetically acceptable carrier comprising a hydrophobic component comprising an oil comprising greater than about 50% polyunsaturated fatty acids. In some embodiments, the oil comprises greater than about 55% polyunsaturated fatty acids. In other embodiments, the oil comprises greater than about 60% polyunsaturated fatty acids. Still further embodiments provide an oil comprising greater than about 65% polyunsaturated fatty acids. Other embodiments provide an oil comprising greater than about 70% polyunsaturated fatty acids. Yet other embodiments provide an oil comprising greater than about 75% polyunsaturated fatty acids. In some embodiments, the oil contains omega-6 and omega-3 polyunsaturated fatty acids. Still further embodiments provide an oil containing a ratio of omega-6 to omega-3 polyunsaturated fatty acids that is greater than about 2:1.

In some embodiments, the oil comprising greater than about 50% polyunsaturated fatty acids is selected from corn oil; cottonseed oil; flaxseed/linseed oil; hemp/hempseed oil; soybean oil; and a combination of two or more thereof. In some embodiments, the oil comprising greater than about 50% polyunsaturated fatty acids comprises hemp/hempseed oil.

In some embodiments, the cosmetically acceptable carrier comprises a surfactant system. In some embodiments, the surfactant system comprises an anionic surfactant, an amphoteric surfactant; a nonionic surfactant; or a combination thereof. In some embodiments, the surfactant system comprises an anionic surfactant. In other embodiments, the anionic surfactant is selected from an alkyl sulfate; an alkyl ether sulfate; and a combination thereof. In some embodiments, the anionic surfactant comprises an alkyl ether sulfate (e.g. laureth-12).

While in other embodiments, the personal care compositions are substantially free of parabens, sulfates, lanolin, and/or harsh preservatives.

In some embodiments, the hydrophobic component further comprises a silicone component. In further embodiments, the silicone component comprises a gel, a powder or a combination thereof.

In certain embodiments, the silicone component comprises polydimethylsiloxane and a silicone crosspolymer. In some embodiments, the polydimethylsiloxane and silicone crosspolymer are present in a weight ratio of less than about 7:1. In some embodiments, the polydimethylsiloxane and silicone crosspolymer are present in a weight ratio of about 4:1.

Yet other embodiments provide personal care compositions comprising from about 0.1 wt. % to about 5 wt. % of a PPAR-α agonist, based on the total weight of the composition. Still further embodiments provide personal care compositions comprising from about 0.2 wt. % to about 2.5 wt. % of a PPAR-α agonist, based on the total weight of the composition. Other embodiments provide personal care compositions comprising from about 0.25 wt. % to about 1 wt. % of a PPAR-α agonist, based on the total weight of the composition. Further embodiments provide personal care compositions comprising from about 0.3 wt. % to about 0.75 wt. % of a PPAR-α agonist, based on the total weight of the composition. In some embodiments, the personal care composition comprises about 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2 wt. %, 2.1 wt. %, 2.2 wt. %, 2.3 wt. %, 2.4 wt. %, 2.5 wt. %, 2.6 wt. %, 2.7 wt. %, 2.8 wt. %, 2.9 wt. %, 3 wt. %, 3.1 wt. %, 3.2 wt. %, 3.3 wt. %, 3.4 wt. %, 3.5 wt. %, 3.6 wt. %, 3.7 wt. %, 3.8 wt. %, 3.9 wt. %, 4 wt. %, 4.1 wt. %, 4.2 wt. %, 4.3 wt. %, 4.4 wt. %, 4.5 wt. %, 4.6 wt. %, 4.7 wt. %, 4.8 wt. %, 4.9 wt. % or 5 wt. % of a PPAR-α agonist, based on the total weight of the composition. In some embodiments, the personal care composition comprises about 0.3 wt. % of a PPAR-α agonist, based on the total weight of the composition.

Some embodiments provide personal care compositions comprising from about 0.1 wt. % to about 10 wt. % of a flavonoid, based on the total weight of the composition. Certain embodiments provide personal care compositions comprising from about 0.2 wt. % to about 8 wt. % of a flavonoid, based on the total weight of the composition. In some embodiments, the personal care composition comprises from about 0.5 wt. % to about 7.5 wt. % of a flavonoid, based on the total weight of the composition. In other embodiments, the personal care composition comprises from about 1 wt. % to about 5 wt. % of a flavonoid, based on the total weight of the composition.

In some embodiments, the hydrophobic component further comprises an oil comprising less than about 50% polyunsaturated fatty acids. In certain embodiments, the personal care composition comprises from about 0.05 wt. % to about 0.5 wt. %, based on the total weight of the composition, of an oil comprising less than about 50% polyunsaturated fatty acids. Yet other embodiments provide personal care compositions comprising from about 0.1 wt. % to about 5 wt. %, based on the total weight of the composition, of an oil comprising greater than about 50% polyunsaturated fatty acids. While further embodiments provide personal care compositions comprising from about 0.5 wt. % to about 4 wt. %, based on the total weight of the composition, of an oil comprising greater than about 50% polyunsaturated fatty acids. In some embodiments, the personal care composition comprises from about 1 wt. % to about 2.5 wt. %, based on the total weight of the composition, of an oil comprising greater than about 50% polyunsaturated fatty acids. Still further embodiments provide personal care compositions comprising from about 1.5 wt. % to about 2 wt. %, based on the total weight of the composition, of an oil comprising greater than about 50% polyunsaturated fatty acids. In some embodiments, the personal care composition comprises about 1.75 wt. %, based on the total weight of the composition, of an oil comprising greater than about 50% polyunsaturated fatty acids.

Other embodiments provide a personal care composition comprising: from about 0.1 wt. % to about 10 wt. %, of a cannabinoid (phytocannabinoid or synthetic); from about 0.1 wt. % to about 5 wt. % of a PPAR-α agonist; from about 1 wt. % to about 2 wt. % of an oil comprising greater than about 50% of polyunsaturated fatty acids; from about 0.1 wt. % to about 20 wt. % of a terpene; from about 0.1 wt. % to about 0.5 wt. % of an oil comprising less than about 50% of polyunsaturated fatty acids; and from about 80 wt. % to about 95 wt. % of a cosmetically acceptable carrier.

Other embodiments provide personal care compositions, wherein the cannabinoid is present in an amount of about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9% about 5 wt. %, 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, about 8 wt. %, about 8.1 wt. %, about 8.2 wt. %, about 8.3 wt. %, about 8.4 wt. %, about 8.5 wt. %, about 8.6 wt. %, about 8.7 wt. %, about 8.8 wt. %, about 8.9 wt. %, about 9 wt. %, about 9.1 wt. %, about 9.2 wt. %, about 9.3 wt. %, about 9.4 wt. %, about 9.5 wt. %, about 9.6 wt. %, about 9.7 wt. %, about 9.8 wt. %, about 9.9% about 10 wt. % based on the total weight of the composition.

In some embodiments, the personal care compositions of the present invention are in a form selected from: a liquid, a solution, an emulsion, a cream, a lotion, a suspension, a triturate, a gel, a jelly, a foam, a paste, an ointment, a shampoo, a salve; a body wash; and a shower gel.

In some embodiments, the personal care compositions of the present invention comprise: Dimethicone, Polysilicone-11, Cyclopentasiloxane, Dimethicone/Vinyl-Dimethicone Crosspolyiner, Laureth-12, Cannabis sativa Seed (Hemp) Seed Oil, an oil w/ <50% polyunsaturated fatty acids (e.g. Persea gratissima oil), Cannabis sativa (Hemp) Oil, Bisabolol, and Paimitoylethanolamide

In other embodiments, the personal care compositions of the present invention comprise: Dimethicone, Polysilicone-11, Cyclopentasiloxane (and) Dimethicone/Vinyl Dimethicone Crosspolymer, Cyclopentasiloxane, of an oil w/ <50% polyunsaturated fatty acids (e.g. Persea gratissima oil), Cannabis sativa (Hemp) Seed Oil, Laureath-12 (and) Polysilicone-11, Cannabis sativa (Hemp) Oil, Bisabolol, and Palmitoylethanolamide.

Still further embodiments provide personal care compositions comprising: Dimethicone, Polysilicone-11, Cyclopentasiloxane, Dimethicone/Vinyl-Dimethicone Crosspolymer, Laureth-12, Cannabis sativa Seed (Hemp) Seed Oil, of an oil w/ <50% polyunsaturated fatty acids (e.g. Persea gratissima oil), Cannabis sativa (Hemp Stems and Stalks) Oil, Bisabolol and Palmitoylethanolamide.

Yet other embodiments provide personal care compositions comprising: Dimethicone, Polysilicone-11, Cyclopentasiloxane (and) Dimethicone/Vin yl Di methicone Crosspolytner, Cyclopentasiloxane, of an oil w/ <50% polyunsaturated fatty acids (e.g. Persea gratissima oil), Cannabis sativa (Hemp) Seed Oil, Laureath-12 (and) Polysilcone-11, Cannabis sativa (Hemp Stems and Stalks) Oil, Bisabolol, and Palmitoylethanolamide.

In certain embodiments, the compositions of the present invention comprise: dimethicone polysilicone-11, caprylyl methicone, dimethicone (and) dimethicone/vinyl dimethicone crosspolymer, dimethyl isosorbide, caprylic/capric triglyceride, of an oil w/ <50% polyunsaturated fatty acids (e.g. Persea gratissima oil), PPG-12/SMDI copolymer, ethoxydiglycol, Cannabis sativa seed (hemp) seed oil, palmitoylethanolamide, Cannabis sativa (hemp) oil, Citrus reticulata (tangerine) peel extracts, oleic acid, cetyl PEG/PPG-10/1 dimethicone, Avena sativa (oat) kernel flour (colloidal oatmeal) and bisabolol.

In other embodiments, the compositions of the present invention comprise: dimethicone polysilicone-11, caprylyl methicone, dimethicone (and) dimethicone/vinyl dimethicone crosspolymer, dimethyl isosorbide, caprylic/capric triglyceride, of an oil w/ <50% polyunsaturated fatty acids (e.g. Persea gratissima oil), PPG-12/SMDI copolymer, ethoxydiglycol, Cannabis sativa (hemp) seed oil, palmitoylethanolamide, Cannabis sativa (hemp) oil, oleic acid, cetyl PEG/PPG-10/1 dimethicone, Avena sativa (oat) kernel flour (colloidal oatmeal) and bisabolol.

In further embodiments, the compositions of the present invention comprise: dimethicone polysilicone-11, caprylyl methicone, dimethicone (and) dimethicone/vinyl dimethicone crosspolymer, dimethyl isosorbide, caprylic/capric triglyceride, PPG-12/SMDI copolymer, ethoxydiglycol, Cannabis sativa (hemp) seed oil, palmitoylethanolamide, Cannabis sativa (hemp) oil, Citrus reticulata (tangerine) peel extracts, oleic acid, cetyl PEG/PPG-10/1 dimethicone, Avena sativa (oat) kernel flour (colloidal oatmeal) and bisabolol.

Still further embodiments provide compositions comprising: dimethicone polysilicone-11, caprylyl methicone, dimethicone (and) dimethicone/vinyl dimethicone crosspolymer, caprylic/capric triglyceride, of an oil w/ <50% polyunsaturated fatty acids (e.g. Persea gratissima oil), PPG-12/SMDI copolymer, ethoxydiglycol, Cannabis sativa (hemp) seed oil, palmitoylethanolamide, Cannabis sativa (hemp) oil, Citrus reticulata (tangerine) peel extracts, oleic acid, cetyl PEG/PPG-10/1 dimethicone, Avena sativa (oat) kernel flour (colloidal oatmeal) and bisaboloi.

Yet other embodiments provide compositions comprising: dimethicone polysilicone-11, caprylyl methicone, dimethicone (and) dimethicone/vinyl dimethicone crosspolymer, dimethyl isosorbide, of an oil w/ <50% polyunsaturated fatty acids (e.g. Persea gratissima oil), PPG-12/SMDI copolymer, ethoxydiglycol, Cannabis sativa (hemp) seed oil, palmitoylethanolamide, Cannabis sativa (hemp) oil, Citrus reticulata (tangerine) peel extracts, oleic acid, cetyl PEG/PPG-10/1 dimethicone, Avena sativa (oat) kernel flour (colloidal oatmeal) and bisabolol.

In certain embodiments, the compositions of the present invention comprise: dimethicone polysilicone-11, caprylyl methicone, dimethicone (and) dimethicone/vinyl dimethicone crosspolymer, dimethyl isosorbide, caprylic/capric triglyceride, of an oil w/ <50% polyunsaturated fatty acids (e.g. Persea gratissima oil), PPG-12/SMDI copolymer, Cannabis sativa (hemp) seed oil, palmitoylethanolamide, Cannabis sativa (hemp) oil, Citrus reticulata (tangerine) peel extracts, cetyl PEG/PPG-10/1 dimethicone, Avena sativa (oat) kernel flour (colloidal oatmeal) and bisaboloi.

In some embodiments, the silicone component comprises dimethicone polysilicone-11, caprylyl methicone, and dimethicone (and) dimethicone/vinyl dimethicone crosspolymer. In some embodiments, the silicone component is substantially free of cyclic silicones. In other embodiments, the silicone component is free of cyclic silicones.

In some embodiments, the compositions of the present invention comprise an oil (e.g. a fractionated coconut oil); a cannabinoid; a PPAR-α agonist; and a surfactant system.

In some embodiments, both or either the cannabinoid(s) and the PPAR-α agonist have undergone mechanical and/or physiochemical modification (micronization) to increase surface area and further reduce molecule particle size substantially below 300 daltons to increase bioavailability and penetration through the skin. Techniques include but are not limited to mechanical milling and grinding, jet milling, and spray drying.

In some embodiments, the compositions of the present invention comprise a hydrophilic wax, a oil w/ >50% polyunsaturated fatty acids (e.g. hemp seed oil) and an oil w/ <50% polyunsaturated fatty acids, optionally further comprising a cannabinoid, a terpene, a fragrance and/or an essential oil.

In some embodiments, the composition comprises from about 1% to about 20% of a hydrophilic wax (e.g. beeswax or a derivative thereof); from about 20% to about 60% of an oil w/ <50% polyunsaturated fatty acids, from about 20% to about 60% of an oil w/ >50% polyunsaturated fatty acids (e.g. hemp seed oil), and from about 0.1% to about 5% of a terpene; and optionally comprising fragrance and/or an essential oil.

In some embodiments, the personal care composition may include a humectant, which is an ingredient that attracts water. The humectant attracts and retains the moisture in the air nearby via absorption, drawing the water vapor into or beneath the surface of the upper layer of the patient's skin. Humectants generally have hydroxyl groups which allow them to participate in hydrogen bonding and attract water. This process attracts moisture from the outer layer of the skin or, in high humidity, from the atmosphere. The moisture is then trapped against the epidermis. Examples of humectants include triethylene glycol, tripropylene glycol, propylene glycol, glycerin, polyethylene glycols, PPG, glycerin, sorbitol, hexylene glycol, butylene glycol, urea, silicones, collagen, aloe, honey, and hyaluronic acid.

In other embodiments, the compositions of the present invention comprise a composition comprising: a humectant (e.g. butylene glycol, glycerin), an acrylic polymer; a surfactant (e.g. decyl glucoside), a plasticizer (e.g. polysorbate), a pH adjusting agent (e.g. sodium hydroxide), a preservative, petrolatum, an oil w/ <50% polyunsaturated fatty acids, a hydrophobic component (e.g. silicones), and emollient, a terpene and water. Still further embodiments provide compositions comprising from about 1% to about 15% of a humectant (e.g. butylene glycol, glycerin); about 0.1% to about 5% of an acrylic polymer; from about 0.1% to about 5% of a surfactant (e.g. decyl glucoside), from about 0.1% to about 5% of a plasticizer (e.g. polysorbate), from about 0.1% to about 5% of a pH adjusting agent (e.g. sodium hydroxide), from about 0.01% to about 1% of a preservative, from about 1% to about 20% of petrolatum, from about 0.1% to about 0.5% of an oil w/<50% polyunsaturated fatty acids, from about 1% to about 30% of a hydrophobic component (e.g. silicones), and from about 0.1% to about 5% of an emollient, from about 0.1% to about 5% of a terpene and from about 60% to about 70% water.

In other embodiments, the compositions of the present invention comprise a composition comprising: a humectant (e.g. butylene glycol, glycerin), an acrylic polymer; a surfactant (e.g. decyl glucoside), a plasticizer (e.g. polysorbate), a pH adjusting agent (e.g. sodium hydroxide), a preservative, petrolatum, an oil w/ >50% polyunsaturated fatty acids (e.g. hemp seed oil), a hydrophobic component (e.g. silicones), and emollient, a terpene and water. Still further embodiments provide compositions comprising from about 1% to about 15% of a humectant (e.g. butylene glycol, glycerin); about 0.1% to about 5% of an acrylic polymer; from about 0.1% to about 5% of a surfactant (e.g. decyl glucoside), from about 0.1% to about 5% of a plasticizer (e.g. polysorbate), from about 0.1% to about 5% of a pH adjusting agent (e.g. sodium hydroxide), from about 0.01% to about 1% of a preservative, from about 1% to about 20% of petrolatum, from about 0.1% to about 0.5% of an oil w/ >50% polyunsaturated fatty acids (e.g. hemp seed oil), from about 1% to about 30% of a hydrophobic component (e.g. silicones), and from about 0.1% to about 5% of an emollient, from about 0.1% to about 5% of a terpene and from about 60% to about 70% water.

Other embodiments include an emollient. An emollient is an ingredient that improves the feel of the lotion on the skin. An emollient may reduce the tackiness and greasiness caused by the other moisturizing ingredients. Examples of common emollients include coconut oil, cetyl esters, selected silicones oils, rhea butter, cocoa butter, mineral oil, lanolin, petrolatum, paraffin, beeswax, squalene, coconut oil, jojoba oil, sesame oil, almond oil, cetyl alcohol, olive oil, oleic acid, and triethylhexanoin.

Still further embodiments comprise an emulsifier. An emulsifier is an ingredient that improves the stability of the mixture of oil materials and water. Examples of common emulsifiers include, glyceryl stearate and stearic acid. Additional examples of emulsifiers include acacia seyal gum octenylsuccinate, C₁₂₋₁₈-acid triglyceride, acrylates crosspolymer-4, Adansonia digitata seed oil peg-8 esters, Adansonia digitata seed oil polyglyceryl-6 esters, almond oil glycereth-8 esters, almond oil peg-6 esters, almond oil peg-8 esters, sweet almond oil peg-8 esters, sweet almond oil polyglyceryl-4 esters, sweet almond oil polyglyceryl-6 esters, aluminum lanolate, ammonium C₆₋₁₆ perfluoroalkylethyl phosphate, ammonium coco-sulfate, ammonium isostearate, ammonium oleate, ammonium perfluorohexyl ethylphosphates, ammonium shellacate, ammonium stearate, ammonium tallate, AMPD-isostearoyl hydrolyzed collagen, AMPD-rosin hydrolyzed collagen, apricot kernel oil peg-6 esters, apricot kernel oil peg-8 esters, apricot kernel oil peg-40 esters, apricot kernel oil polyglyceryl-3 esters, apricot kernel oil polyglyceryl-4 esters, apricot kernel oil polyglyceryl-5 esters, apricot kernel oil polyglyceryl-6 esters, apricot kernel oil polyglyceryl-10 esters, arachideth-20, arachidic acid, arachidyl glucoside, argan oil peg-8 esters, argan oil polyglyceryl-6 esters, astrocaryum vulgare oil polyglyceryl-6 esters, avocadamide DEA, avocadamide DIPA, avocado oil peg-8 esters, avocado oil propylene glycol esters, babassu oil glycereth-8 esters, babassu oil polyglyceryl-6 esters, babassuamidopropyltrimonium chloride, babassuamidopropyltrimonium methosulfate, behenamidopropyl dimethylamine, beheneth-2, beheneth-5, beheneth-10, beheneth-15, beheneth-20, beheneth-25, beheneth-30, behenoyl stearic acid, behenyl PG-trimonium chloride, benzenesulfonyltromethamide, bertholletia excelsa seed oil polyglyceryl-6 esters, borage seed oil peg-8 esters, borage seed oil polyglyceryl-4 esters, borage seed oil polyglyceryl-6 esters, butoxynol-19 carboxylic acid, butoxynol-5 carboxylic acid, butyl octanoic acid, bis-iso butyl peg/ppg-10/7/dimethicone copolymer, bis-butyldimethicone polyglyceryl-3, butyldimoniumhydroxypropyl butylglucosides chloride, butyldimoniumhydroxypropyl laurylglucosides chloride, butylene glycol behenate, butylene glycol cocoate, butylene glycol isostearate, butylene glycol isostearates, butylene glycol laurate, butylene glycol myristate, butylene glycol stearate, butylglucoside caprate, butylglucosides hydroxypropyltrimonium chloride, C₁₋₅ alkyl galactomannan, C₁₀₋₁₆ alkyl glucoside, C₁₀₋₁₈ triglycerides polyglyceryl-3 esters phosphates, C₁₀₋₃₀ cholesterol/lanosterol esters, C₁₀₋₄₀ isoalkyl acid glyceride, C₁₁₋₁₃ pareth-6, C₁₁₋₁₃ pareth-9, C₁₁₋₁₃ pareth-10, C₁₁₋₁₅ pareth-3, C₁₁₋₁₅ pareth-5, C₁₁₋₁₅ pareth-7, C₁₁₋₁₅ pareth-9, C₁₁₋₁₅ pareth-12, C₁₁₋₁₅ pareth-15, C₁₁₋₁₅ pareth-20, C₁₁₋₁₅ pareth-30, C₁₁₋₁₅ pareth-3 oleate, C₁₁₋₁₅ pareth-7 carboxylic acid, C₁₁₋₁₅ sec-pareth-12, C₁₁₋₂₁ pareth-3, C₁₁₋₂₁ pareth-10, C₁₂₋₁₃ pareth-2, C₁₂₋₁₃ pareth-3, C₁₂₋₁₃ pareth-4, C₁₂₋₁₃ pareth-5, C₁₂₋₁₃ pareth-6, C₁₂-13 pareth-7, C₁₂₋₁₃ pareth-9, C₁₂₋₁₃ pareth-10, C₁₂₋₁₃ pareth-15, C₁₂₋₁₃ pareth-23, C₁₂₋₁₃ pareth-10 phosphate, C₁₂₋₁₄ alkyl diaminoethylglycine HCl, C₁₂₋₁₄ pareth-7, C₁₂₋₁₄ pareth-12, C₁₂₋₁₄ pareth-3, C₁₂₋₁₄ sec-pareth-3, C₁₂₋₁₄ sec-pareth-5, C₁₂₋₁₄ sec-pareth-7, C₁₂₋₁₄ sec-pareth-8, C₁₂₋₁₄ sec-pareth-9, C₁₂₋₁₄ sec-pareth-12, C₁₂₋₁₄ sec-pareth-15, C₁₂₋₁₄ sec-pareth-20, C₁₂₋₁₄ sec-pareth-30, C₁₂₋₁₄ sec-pareth-40, C₁₂₋₁₄ sec-pareth-50, C₁₂₋₁₅ pareth-2, C₁₂₋₁₅ pareth-2 phosphate, C₁₂₋₁₅ pareth-3 pareth-12, C₁₂₋₁₅ phosphate, C₁₂₋₁₅ pareth-4, C₁₂₋₁₅ pareth-5, C₁₂₋₁₅ pareth-6 phosphate, C₁₂₋₁₅ pareth-8 carboxylic acid, C₁₂₋₁₅ pareth-8 phosphate, C₁₂₋₁₅ pareth-9 phosphate, C₁₂₋₁₅ pareth-10, C₁₂₋₁₅ pareth-10 phosphate, C₁₂₋₁₅ Pareth-11, C₁₂₋₁₅ pareth-3, C₁₂₋₁₅ pareth-7, C₁₂₋₁₅ pareth-9, C₁₂₋₁₅ pareth-9 hydrogenated tallowate, C₁₂₋₁₆ pareth-5, C₁₂₋₁₆ pareth-6 phosphate, C₁₂₋₁₆ pareth-7, C₁₂₋₁₆ pareth-9, C₁₂₋₂₀ acid peg-8 ester, C₁₂₋₂₀ alkyl glucoside, C₁₄₋₁₅ pareth-4, C₁₄₋₁₅ pareth-7, C₁₄₋₁₅ pareth-11, C₁₄₋₁₅ pareth-12, C₁₄₋₁₅ pareth-13, C₁₄₋₁₆ glycol palmitate, C₁₈₋₂₀ glycol isostearate, C₁₈₋₃₆ acid, C₁₈₋₃₆ acid glycol ester, C₂₀₋₂₂ alkyl phosphate, C₂₀₋₂₂ pareth-30, C₂₀₋₄₀ alcohols, C₂₀₋₄₀ pareth-3, C₂₀₋₄₀ pareth-10, C₂₀₋₄₀ pareth-24, C₂₀₋₄₀ pareth-40, C₂₂₋₂₄ pareth-33, C₂₉₋₇₀ acid, C₃₀₋₃₈ olefin/isopropyl maleate/MA copolymer, C₃₀₋₅₀ pareth-3, C₃₀₋₅₀ pareth-10, C₃₀₋₅₀ pareth-40, C₄₀₋₆₀ acid, C₄₀₋₆₀ alcohols, C₄₀₋₆₀ pareth-3, C₄₀₋₆₀ pareth-10, C₆₋₁₀ pareth-4 phosphate, C₆₋₈ alkyl C₃₋₆ alkyl glucoside dimethicone, C₉₋₁₁ pareth-4, C₉₋₁₁ pareth-6, C₉₋₁₁ pareth-8, C₉₋₁₁ pareth-3, C₉₋₁₅ alkylphosphate, C₉₋₁₅ pareth-8, C₉₋₁₆ alkane/cycloalkane, calcium stearoyl lactylate, candelilla/jojoba/rice bran polyglyceryl-3 esters, hydrolyzed Candida bombicola sophorolipids, Cannabis sativa seed oil peg-8 esters, capryl/lauryl wheat bran/straw glycosides, capryleth-4, capryleth-4 carboxylic acid, capryleth-5, capryleth-6 carboxylic acid, capryleth-9 carboxylic acid, caprylic/capric glycerides polyglycerin-10 esters, caprylic/capric triglyceride peg-4 esters, caprylic/capric/succinic triglyceride, caprylyl dimethicone ethoxy glucoside, caprylyl/capryl wheat bran/straw glycosides, caprylyl/capryl xylosides, carapa guaianensis oil peg-8 esters, carpotroche brasiliensis seed acid, hydrogenated castor oil, cera alba wax, ceteareth-2, ceteareth-3, ceteareth-4, ceteareth-5, ceteareth-6, ceteareth-7, ceteareth-8, iso ceteareth-8 stearate, ceteareth-9, ceteareth-10, ceteareth-11, ceteareth-12, ceteareth-13, ceteareth-14, ceteareth-15, ceteareth-16, ceteareth-17, ceteareth-18, ceteareth-20, ceteareth-22, ceteareth-23, ceteareth-24, ceteareth-25, ceteareth-27, ceteareth-28, ceteareth-29, ceteareth-30, ceteareth-33, ceteareth-34, ceteareth-50, ceteareth-60, ceteareth-6 olivate, ceteareth-60 myristyl glycol, cetearyl glucoside, cetearyl palmate, cetearyl wheat bran glycosides, cetearyl wheat straw glycosides, ceteth-1, ceteth-2, ceteth-3, ceteth-4, ceteth-5, iso ceteth-5, ceteth-6, ceteth-7, iso ceteth-7, ceteth-8, ceteth-8 phosphate, ceteth-10, iso ceteth-10, iso ceteth-10 stearate, ceteth-12, iso ceteth-12, ceteth-13, ceteth-14, ceteth-15, iso ceteth-15, ceteth-16, ceteth-17, ceteth-18, ceteth-20, isoceteth-20, ceteth-23, ceteth-24, ceteth-25, iso ceteth-25, ceteth-30, iso ceteth-30, cetoleth-2, cetoleth-4, cetoleth-5, cetoleth-6, cetoleth-10, cetoleth-11, cetoleth-15, cetoleth-18, cetoleth-20, cetoleth-22, cetyl glyceryl ether/glycerin copolymer, cetyl peg-8 dimethicone, cetyl peg/ppg-10/1 dimethicone, cetyl peg/ppg-15/15 butyl ether dimethicone, cetyl peg/ppg-7/3 dimethicone, cetyl phosphate, cetyl pyridinium chloride anhydrous, chimyl stearate, chitosan lauramide hydroxypropyltrimonium chloride, chitosan PCA palmitamide succinamide, chitosan stearamide hydroxypropyltrimonium chloride, cholesterol, cholesteryl C16-18 alkenyl succinate, choleth-5, choleth-10, choleth-15, choleth-20, choleth-24, choleth-30, cocamide DIPA, cocamine, coceth-3, coceth-5, coceth-6, coceth-7, coceth-7 carboxylic acid, coceth-8, coceth-10, coceth-20, coceth-25, coco-oleate estolides, coco/sunfloweramidopropyl betaine, cocoa butter glyceryl esters, cocoglucosides hydroxypropyltrimonium chloride, cocoglycerides, coconut acid, hydrogenated coconut oil peg-10 esters, coconut oil polyglyceryl-6 esters, coconut oil ppg-2-peg-6 esters, cocoyl ethyl glucoside, coffee seed oil peg-8 esters, coffee seed oil polyglyceryl-6 esters, corn oil glycereth-8 esters, acetylated hydrogenated cottonseed glyceride, Crambe abyssinica seed oil phytosterol esters, deceth-3, deceth-4, iso deceth-4, deceth-4 phosphate, deceth-5, isodeceth-5, deceth-6, isodeceth-6, deceth-6 phosphate, deceth-7, deceth-8, deceth-9, deceth-10, isodeceth-2 cocoate, decyltetradeceth-5, decyltetradeceth-15, decyltetradeceth-20, decyltetradeceth-25, decyltetradeceth-30, decyltetradeceth-10, di-C₁₂₋₁₅ pareth-2 phosphate, di-C₁₂₋₁₅ pareth-4 phosphate, di-C₁₂₋₁₅ pareth-6 phosphate, di-C₁₂₋₁₅ pareth-8 phosphate, di-C₁₂₋₁₅ pareth-10 phosphate, dibehenyl/diarachidyl dimonium chloride, dibehenyldimonium methosulfate, dibutoxymethane, dicapryl/dicaprylyl dimonium chloride, diceteareth-10 phosphate, dicocodimethylamine dilinoleate, didodecyl butanetetracarboxylate, diethyl aminoethyl cocoate, diethylaminoethyl peg-5 laurate, diethylene glycol diisononanoate, digalactosyl glyceryl linoleate/palmitate/oleate, diglycerin/dilinoleic acid/hydroxystearic acid copolymer, diglyceryl sorbitan tetraethylhexanoate, dihydrocholeth-15, dihydrocholeth-20, dihydrocholeth-30, dihydrolanosterol, dihydroxyethyl tallowamine oxide, dilaureth-4 phosphate, dilaureth-7 citrate, dilaureth-10 phosphate, hydrogenated dimer dilinoleth-20, hydrogenated dimer dilinoleth-30, hydrogenated dimer dilinoleth-40, hydrogenated dimer dilinoleth-80, hydrogenated dimer dilinoleth-60, dimethicone peg-7 phosphate, dimethicone peg-7 undecylenate, dimethicone peg-8 avocadoate, dimethicone peg-10 phosphate, dimethicone peg-15 acetate, dimethicone peg/ppg-12/4 phosphate, dimethicone peg/ppg-7/4 phosphate, dimethicone/peg-10 crosspolymer, dimethicone/polyglycerin-3 crosspolymer, dimethoxysilyl ethylenediaminopropyl dimethicone, dimethyl octynediol, dimethyl soyamine, dimethyl stearamine, dimethyl tallowamine, dimyristyl phosphate, dinonoxynol-4 phosphate, dinonoxynol-9 citrate, dioctyldodecyl stearoyl glutamate, dioleth-8 phosphate, dipentaerythrityl hexaheptanoate/hexacaprylate/hexacaprate, dipentaerythrityl hexahydroxystearate, disodium babassuamido MEA-sulfosuccinate, disodium cetyl phenyl ether disulfonate, disodium coceth-3 sulfosuccinate, disodium coco-glucoside citrate, disodium coco-glucoside sulfosuccinate, disodium decyl phenyl ether disulfonate, disodium laureth-7 citrate, disodium lauroamphodiacetate lauroyl sarcosinate, disodium oleyl phosphate, disodium peg-8 glyceryl caprylate/caprate, disodium peg-8 ricinosuccinate, disodium sitostereth-14 sulfosuccinate, distearamidopropylmethylamine, dodecylhexadecyltrimonium chloride, dodoxynol-13, ethyl hydroxyethyl cellulose, ethylene dihydrogenated tallowamide, hydrolyzed ethylene/MA copolymer, ethylhexyl coco-oleate estolide, ethylhexyl linoleoyl stearate, ethylhexylglyceryl behenate, ethylhexylglyceryl palmitate, ethylhexyloxyglyceryl palmitate, fluoro C2-8 alkyldimethicone, perfluorononylethyl carboxydecyl peg-10 dimethicone, fusel wheat bran/straw glycosides, glycereth-5 cocoate, glycereth-5 lactate, glycereth-6 laurate, glycereth-8 hydroxystearate, glycereth-20 stearate, glycereth-17 cocoate, glycereth-17 tallowate, glycereth-25 PCA isostearate, glyceryl arachidate, glyceryl arachidonate, glyceryl behenate, glyceryl behenate/eicosadioate, glyceryl capryl ether, glyceryl caprylate, glyceryl caprylate/caprate, glyceryl citrate/lactate/linoleate/oleate, glyceryl cocoate, glyceryl dilaurate, glyceryl dipalmitate, glyceryl erucate, glyceryl glycyrrhetinate, glyceryl hydrogenated rapeseedate, glyceryl hydroxystearate, glyceryl isopalmitate, glyceryl isostearate, glyceryl isostearates, glyceryl isotridecanoate/stearate/adipate, glyceryl laurate, 1-glyceryl laurate, glyceryl laurate SE, glyceryl lauryl ether, glyceryl linoleate, (S)-glyceryl linoleate, glyceryl montanate, glyceryl myristate, glyceryl oleate citrate, glyceryl oleate SE, glyceryl oleate/elaidate, glyceryl palmitate lactate, glyceryl palmitoleate, glyceryl pentadecanoate, glyceryl ricinoleate SE, glyceryl sesquioleate, glyceryl stearate citrate, glyceryl stearate lactate, glyceryl stearate SE, glyceryl stearate/maleate, glyceryl tallowate, glyceryl undecylenate, glyceryl/sorbitol oleate/hydroxystearate, glycol cetearate, glycol ethylhexanoate, hexacosyl glycol isostearate, 2-hexyl decanoic acid, hexyldeceth-2, hexyldeceth-20, hydroxycetyl isostearate, hydroxycetyl phosphate, hydroxyethyldiethonium polyisobutenyl triethylaminosuccinate, hydroxypropylcocoate peg-8 dimethicone, hydroxystearyl alcohol, hydroxystearyl glucoside, hydroxysuccinimidyl C10-40 isoalkyl acidate, laneth-4 phosphate, laneth-5, laneth-10, laneth-10 acetate, laneth-16, hydrogenated laneth-20, laneth-25, hydrogenated laneth-25, laneth-50, laneth-75, lard glyceride, hydrogenated lard glyceride, laurdimonium chlorohydroxypropyl chloride, laurdimoniumhydroxypropyl cocoglucosides chloride, laurdimoniumhydroxypropyl decylglucosides chloride, laurdimoniumhydroxypropyl laurylglucosides chloride, laureth-1, laureth-2, laureth-3, isolaureth-3, laureth-3 carboxylic acid, laureth-4, laureth-4 phosphate, isolaureth-4 phosphate, laureth-5, laureth-5 butyl ether, laureth-5 carboxylic acid, laureth-6, isolaureth-6, laureth-7, laureth-7 phosphate, laureth-8, laureth-9, laureth-10, isolaureth-10, laureth-11, laureth-12, laureth-13, laureth-14, laureth-15, laureth-16, laureth-20, laureth-23, laureth-25, laureth-30, laureth-13 pg-hydroxyethylcellulose, laurimino bispropanediol, lauroyl ethyl glucoside, lauryl dimethicone peg-10 phosphate, lauryl dimethicone peg-15 crosspolymer, lauryl isopentyl-peg/ppg-18/18 methicone, lauryl peg-10 tris(trimethylsiloxy)silylethyl dimethicone, lauryl peg-8 ppg-8 dimethicone, lauryl phosphate, lauryl polyglyceryl-6 cetearyl glycol ether, lauryl sucrose, lauryl/myristyl wheat bran/straw glycosides, lecithin, hydroxylated lecithin, linseed acid, linseed oil polyglyceryl-4 esters, lithium myristate, lysolecithin, hydrogenated lysolecithin, hydrogenated lysophosphatidylcholine, lysophosphatidylglycerol, magnesium coceth sulfate, mannitan laurate, mannitan oleate, mannosylerythritol lipid, Mauritia flexuosa seed oil peg-8 esters, Mauritia flexuosa seed oil polyglyceryl-6 esters, MEA-laureth-6 carboxylate, MEA-ppg-8-steareth-7 carboxylate, hydrogenated menhaden acid, methoxy peg-17/dodecyl glycol copolymer, bis-methoxy peg-40 polyepsilon caprolactone, methoxy peg/ppg-25/4 dimethicone, methyl gluceth-10, methyl glucose isostearate, methyl glucose laurate, methyl glucose sesquicaprylate/sesquicaprate, methyl glucose sesquicocoate, methyl glucose sesquiisostearate, methyl glucose sesquilaurate, methyl glucose sesquioleate, methyl glucose sesquistearate, methyl isostearate, methyl silanol peg-7 glyceryl cocoate, methyl silanol tri-peg-8 glyceryl cocoate, methylglucose dioleate/hydroxystearate, minkamidopropyl dimethylamine, myreth-2, myreth-3, myreth-4, myreth-5, isomyreth-9, myreth-10, isomyreth-3, myristoyl ethyl glucoside, myristoyl lactylic acid, myristyldimoniumhydroxypropyl cocoglucosides chloride, nonyl nonoxynol-5, oatamidopropyl dimethylamine, octadecenedioic acid, octyldecyl oleate, octyldecyl phosphate, octyldodeceth-2, octyldodeceth-5, octyldodeceth-10, octyldodeceth-16, octyldodeceth-20, octyldodeceth-25, octyldodecyl glucoside, octyldodecyl xyloside, oleamide DIPA, (Z)-oleic acid, oleoyl ethyl glucoside, oleth-10, oleth-11, oleth-12, oleth-15, oleth-16, oleth-45, oleth-2, oleth-2 phosphate, oleth-20 phosphate, oleth-3, oleth-30, oleth-35, oleth-4, oleth-40, oleth-5, oleth-6, oleth-7, oleth-8, oleth-82, oleth-9, oleyl phosphate, olivamidopropyltrimonium chloride, olive glycerides, olive oil glycereth-8 esters, olive oil peg-7 esters, olive oil peg-8 esters, olive oil peg-10 esters, olive oil peg/ppg-3/1 esters, olive oil polyglyceryl-6 esters, olive oil polyglyceryl-6 esters, palm acid, palm glyceride, hydrogenated palm glycerides, hydrogenated palm glycerides, palm kernel acid, palm kernel alcohol, palm kernel glycerides, acetylated palm kernel glycerides, palm kernel oil polyglyceryl-4 esters, palm kernelamide MIPA, hydrogenated palm oil, palm oil glycereth-8 esters, palm oil peg-8 esters, palm oil polyglyceryl-3 esters, palm oil polyglyceryl-4 esters, palm oil polyglyceryl-6 esters, hydrogenated palm/palm kernel glycerides, hydrogenated palm/palm kernel oil peg-6 esters, palmamide DEA, palmamide MEA, palmamide MIPA, palmeth-2, palmeth-2 phosphate, palmitamidobutyl guanidine acetate, palmitic acid, palmitoyl ethyltrimonium methosulfate, palmitoyl inulin, parinari curatellifolia oil peg-8 esters, parinari curatellifolia oil polyglyceryl-6 esters, peanut glycerides, peanutamide MIPA, peg-2 caprylylamine, peg-2 cocamide, peg-2 cocamine, peg-2 diisostearate, peg-2 dioleate, peg-2 hydrogenated castor oil, peg-2 hydrogenated tallow amine, peg-2 isostearate, peg-2 lauramide, peg-2 oleate SE, peg-2 olive glycerides, peg-2 sorbitan isostearate, peg-2 sorbitan trioleate, peg-2 soyamine, peg-2 stearate SE, peg-2 sunflower glycerides, peg-2 tallowamide DEA, peg-3 butylene glycol laurate, peg-3 castor oil, peg-3 cocamide DEA, peg-3 cocamine, peg-3 dicaprylate/caprate, peg-3 diisostearate, peg-3 distearate, peg-3 glyceryl cocoate, peg-3 glyceryl isostearate, peg-3 glyceryl triisostearate, peg-3 glyceryl trioleate, peg-3 glyceryl tristearate, peg-3 isostearate, peg-3 lanolate, peg-3 lauramide, peg-3 oleamide, peg-3 oleate, peg-3 sorbitan oleate, peg-3 sorbitan stearate, peg-3 sorbitan tristearate, peg-3 stearate, peg-3 tallow aminopropylamine, peg-3/ppg-2 glyceryl/sorbitol hydroxystearate/isostearate, peg-4 caprylic/capric glycerides, peg-4 castor oil, peg-4 cocamide, peg-4 cocamine, peg-4 diisostearate, peg-4 ethylhexanoate, peg-4 glyceryl tristearate, peg-4 isostearate, peg-4 lanolate, peg-4 oleate, peg-4 polyglyceryl-2 distearate, peg-4 polyglyceryl-2 stearate, peg-4 sorbitan stearate, peg-4 sorbitan triisostearate, peg-4 stearate, peg-4 tallate, peg-4 trimethylolpropane distearate, peg-5 castor oil, peg-5 cocamine, peg-5 glyceryl isostearate, peg-5 glyceryl oleate, peg-5 glyceryl stearate, peg-5 glyceryl triisostearate, peg-5 glyceryl trioleate, peg-5 glyceryl tristearate, peg-5 hydrogenated castor oil, peg-5 hydrogenated castor oil isostearate, peg-5 hydrogenated castor oil triisostearate, peg-5 hydrogenated corn glycerides, peg-5 hydrogenated lanolin, peg-5 lanolate, peg-5 lanolin, peg-5 lanolinamide, peg-5 lauramide, peg-5 oleamide dioleate, peg-5 oleamine, peg-5 oleate, peg-5 phytosterol, peg-5 rapeseed sterol, peg-5 soy sterol, peg-5 soyamine, peg-5 stearamine, peg-5 stearate, peg-5 tallate, peg-5 tricapryl citrate, peg-5 trimethylolpropane trimyristate, peg-5 tsubakiate glycerides, peg-6 almond glycerides, peg-6 beeswax, peg-6 butylene glycol laurate, peg-6 caprylate/caprate, peg-6 diisostearate, peg-6 dioleate, peg-6 glyceryl caprate, peg-6 glyceryl isostearate, peg-6 glyceryl tristearate, peg-6 hydrogenated castor oil, peg-6 hydrogenated palm/palm kernel glyceride, peg-6 hydrogenated palmamide, peg-6 isopalmitate, peg-6 isostearate, peg-6 lanolate, peg-6 lauramide, peg-6 laurate, peg-6 laurate/tartrate, peg-6 methicone acetate, peg-6 methyl ether dimethicone, peg-6 oleamine, peg-6 oleate, peg-6 olive glycerides, peg-6 propylene glycol caprylate/caprate, peg-6 sorbitan oleate, peg-6 sorbitan stearate, peg-6 stearate, peg-6 stearylguanidine, peg-7 caprylic/capric glycerides, peg-7 cocamide, peg-7 cocoglycerides, peg-7 glyceryl soyate, peg-7 hydrogenated castor oil, peg-7 lanolate, peg-7 methyl ether dimethicone, peg-7 oleate, peg-7 olive glycerides, peg-7 ricinoleamide, peg-7 sunflower glycerides, peg-7 tallow amine, peg-7 trimethylolpropane coconut ether, peg-8 avocadoate, peg-8 behenate, peg-8 C12-18 alkyl ester, peg-8 caprate, peg-8 caprylate, peg-8 caprylate/caprate, peg-8 caprylic/capric glycerides, peg-8 castor oil, peg-8 cocamine, peg-8 cranberriate, peg-8 di/triricinoleate, peg-8 dicocoate, peg-8 diisostearate, peg-8 dodecenylsuccinate, peg-8 glyceryl isostearate, peg-8 hydrogenated castor oil, peg-8 hydrogenated fish glycerides, peg-8 isostearate, peg-8 lanolate, peg-8 myristate, peg-8 oleate, peg-8 peg-4 dimethicone, peg-8 raspberriate, peg-8 sesquilaurate, peg-8 sesquioleate, peg-8 soyamine, peg-8 tallate, peg-8 trisiloxane, peg-8 undecylenate, peg-9 avocadoate, peg-9 borageate, peg-9 butylene glycol laurate, peg-9 butyloctanoate, peg-9 castor oil, peg-9 cocoglycerides, peg-9 diethylmonium chloride, peg-9 glyceryl isostearate, peg-9 grapeseedate, peg-9 isostearate, peg-9 laurate, peg-9 methyl ether dimethicone, peg-9 octyldodecanoate, peg-9 oleate, peg-9 polydimethylsiloxyethyl dimethicone, peg-9 soyate, peg-9 stearate, peg-10 butylene glycol isostearate, peg-10 castor oil, peg-10 cocamine, peg-10 cocoate, peg-10 dioleate, peg-10 glyceryl diisostearate, peg-10 glyceryl isostearate, peg-10 glyceryl oleate, peg-10 glyceryl stearate, peg-10 glyceryl triisostearate, peg-10 glyceryl trioleate, peg-10 glyceryl tristearate, peg-10 hydrogenated castor oil, peg-10 hydrogenated castor oil isostearate, peg-10 hydrogenated castor oil triisostearate, peg-10 hydrogenated lanolin, peg-10 hydrogenated tallow amine, peg-10 isolauryl thioether, peg-10 isostearate, peg-10 lanolate, peg-10 lanolin, peg-10 laurate, peg-10 methyl ether dimethicone, peg-10 oleamine, peg-10 oleate, peg-10 olive glycerides, peg-10 phytosterol, peg-10 rapeseed sterol, peg-10 sorbitan laurate, peg-10 soy sterol, peg-10 soyamine, peg-10 stearamide, peg-10 stearate, peg-10 sunflower glycerides, peg-10 tallate, peg-10 tallow aminopropyl amine, peg-10 tsubakiate glycerides, peg-11 avocado glycerides, peg-11 babassu glycerides, peg-11 castor oil, peg-11 cocamide, peg-11 cocoa butter glycerides, peg-11 lauramide, peg-11 methyl ether dimethicone, peg-11 oleate, peg-11 tallow amine, peg-12 beeswax, peg-12 cocamine, peg-12 diisostearate, peg-12 dilaurate, peg-12 dimethicone crosspolymer, peg-12 dioleate, peg-12 distearate, peg-12 ditallate, peg-12 glyceryl laurate, peg-12 isostearate, peg-12 lanolate, peg-12 laurate, peg-12 oleate, peg-12 palm kernel glycerides, peg-12 stearate, peg-12 tallate, peg-13 diphenylol propane, peg-13 ethylhexanoate, peg-13 hydrogenated tallow amide, peg-13 sunflower glycerides, peg-14 avocado glycerides, peg-14 laurate, peg-14 oleate, peg-14 stearate, peg-14 tallate, peg-15 butanediol, peg-15 butylene glycol diisostearate, peg-15 castor oil, peg-15 cocamine, peg-15 cocamine oleate/phosphate, peg-15 cocoate, peg-15 cocopolyamine, peg-15 glyceryl diisostearate, peg-15 glyceryl laurate, peg-15 glyceryl oleate, peg-15 glyceryl ricinoleate, peg-15 glyceryl stearate, peg-15 glyceryl triisostearate, peg-15 glyceryl trioleate, peg-15 glyceryl tristearate, peg-15 hydrogenated castor oil isostearate, peg-15 hydrogenated castor oil triisostearate, peg-15 hydrogenated lanolin, peg-15 hydrogenated tallow amine, peg-15 hydroxystearate, peg-15 jojoba acid, peg-15 jojoba alcohol, peg-15 lanolate, bis-peg-15 methyl ether dimethicone, peg-15 oleamine, peg-15 oleate, peg-15 phytosterol, peg-15 soyamine, peg-15 stearamide, peg-15 stearamine, peg-15 stearate, peg-15 tallate, peg-15 tallow aminopropyl amine, peg-16 castor oil, peg-16 cetyl/oleyl/stearyl/lanolin alcohol ether, peg-16 dilaurate, peg-16 hydrogenated castor oil, peg-16 macadamia glycerides, peg-16 oleate, peg-16 soy sterol, peg-16 tallate, peg-18 castor oil dioleate, peg-18 glyceryl oleate/cocoate, peg-18 palm glycerides, peg-18 stearate, peg-20 almond glycerides, peg-20 beeswax, peg-20 castor oil, peg-20 cocamide, peg-20 cocamine, peg-20 corn glycerides, peg-20 dioleate, peg-20 evening primrose glycerides, peg-20 glyceryl diisostearate, peg-20 glyceryl triisostearate, peg-20 glyceryl trioleate, peg-20 glyceryl tristearate, peg-20 hexadecenylsuccinate, peg-20 hydrogenated castor oil, peg-20 hydrogenated castor oil isostearate, peg-20 hydrogenated castor oil PCA isostearate, peg-20 hydrogenated dimer dilinoleate, peg-20 hydrogenated palm glycerides, peg-20 isostearate, peg-20 lanolate, peg-20 lanolin, peg-20 mannitan laurate, peg-20 methyl glucose sesquicaprylate/sesquicaprate, peg-20 methyl glucose sesquilaurate, peg-20 myristate, peg-20 oleamine, peg-20 oleate, peg-20 sorbitan isostearate, peg-20 sorbitan oleate, peg-20 sorbitan triisostearate, peg-20 soy sterol, peg-20 tallate, peg-20 tsubakiate glycerides, peg-20-ppg-10 glyceryl stearate, peg-23 hexadecyleicosanoate, peg-23 octyldodecanoate, peg-23 oleate, peg-23 stearate, peg-24 hydrogenated lanolin, peg-24 lanolin, peg-25 butylene glycol isostearate, peg-25 glyceryl isostearate, peg-25 glyceryl trioleate, peg-25 hydrogenated castor oil, peg-25 moringa glycerides, peg-25 phytostanol, peg-25 propylene glycol stearate, peg-25 soy sterol, peg-25 stearate, peg-26 castor oil, peg-26 jojoba acid, peg-26 jojoba alcohol, peg-27 lanolin, peg-30 castor oil, peg-30 glyceryl diisostearate, peg-30 glyceryl triisostearate, peg-30 glyceryl trioleate, peg-30 hydrogenated castor oil, peg-30 hydrogenated castor oil isostearate, peg-30 hydrogenated castor oil PCA isostearate, peg-30 hydrogenated castor oil triisostearate, peg-30 hydrogenated dimer dilinoleate, peg-30 hydrogenated lanolin, peg-30 isostearate, peg-30 lanolin, peg-30 oleamine, peg-30 phytosterol, peg-30 sorbitan tetraoleate, peg-30 soy sterol, peg-32 dioleate, peg-32 methyl ether dimethicone, peg-32 oleate, peg-32 stearate, peg-33 castor oil, peg-35 almond glycerides, peg-35 hydrogenated castor oil, peg-35 lanolin, peg-35 soy glycerides, peg-35 stearate, peg-36 castor oil, peg-36 oleate, peg-36 stearate, peg-40 distearate, peg-40 glyceryl isostearate, peg-40 glyceryl stearate, peg-40 glyceryl triisostearate, peg-40 glyceryl trioleate, peg-40 hydrogenated castor oil isostearate, peg-40 hydrogenated castor oil triisostearate, peg-40 hydrogenated dimer dilinoleate, peg-40 hydrogenated lanolin, peg-40 isostearate, peg-40 jojoba acid, peg-40 jojoba alcohol, peg-40 lanolin, peg-40 olive glycerides, peg-40 ricinoleamide, peg-40 sorbitan diisostearate, peg-40 sorbitan laurate, peg-40 sorbitan oleate, peg-40 sorbitan perisostearate, peg-40 sorbitan stearate, peg-40 sorbitan tetraoleate, peg-40 soy sterol, peg-42 babassu glycerides, peg-42 mushroom glycerides, peg-44 castor oil, peg-45 hydrogenated castor oil, peg-45 palm kernel glycerides, peg-45 safflower glycerides, peg-45 stearate, peg-50 castor oil, peg-50 glyceryl isostearate, peg-50 glyceryl triisostearate, peg-50 glyceryl trioleate, peg-50 hydrogenated castor oil isostearate, peg-50 hydrogenated castor oil succinate, peg-50 hydrogenated castor oil triisostearate, peg-50 lanolin, peg-50 stearamide, peg-50 stearamine, peg-54 castor oil, peg-54 hydrogenated castor oil, peg-55 castor oil, peg-55 hydrogenated castor oil, peg-55 lanolin, peg-55 stearate, peg-58 hydrogenated castor oil isostearate, peg-60 castor oil, peg-60 castor oil isostearate, peg-60 corn glycerides, peg-60 evening primrose glycerides, peg-60 glyceryl diisostearate, peg-60 glyceryl stearate, peg-60 glyceryl triisostearate, peg-60 glyceryl trioleate, peg-60 hydrogenated castor oil, peg-60 hydrogenated castor oil triisostearate, peg-60 hydrogenated dimer dilinoleate, peg-60 lanolin, peg-60 Passiflora edulis seed glycerides, peg-60 Passiflora incarnata seed glycerides, peg-60 rhea butter glycerides, peg-60 sorbitan tetraoleate, peg-60 sorbitan tetrastearate, peg-60 tsubakiate glycerides, peg-65 butylene glycol isostearate, peg-65 hydrogenated castor oil, peg-66 trihydroxystearin, peg-70 hydrogenated lanolin, peg-70 mango glycerides, peg-75 beta-sitosterol, peg-75 castor oil, peg-75 cocoa butter glycerides, peg-75 Crambe abyssinica seed oil, peg-75 dioleate, peg-75 lanolin, peg-75 lanolin wax, peg-75 meadowfoam oil, peg-75 propylene glycol stearate, peg-75 shea butter glycerides, peg-75 chorea butter glycerides, peg-75 soy glycerides, peg-80 castor oil, peg-80 hydrogenated castor oil, peg-80 hydrogenated dimer dilinoleate, peg-80 hydrogenated glyceryl palmate, peg-80 methyl glucose laurate, peg-80 sorbitan palmitate, peg-82 glyceryl tallowate, peg-85 lanolin, peg-90/polyepsilon caprolactone, peg-100 castor oil, peg-100 hydrogenated castor oil, peg-100 lanolin, peg-114 polylactic acid, peg-12 glyceryl dioleate, peg-12 palmitamine, peg-120 distearate, peg-120 methyl glucose dioleate, peg-120 propylene glycol stearate, peg-130 glyceryl tallowate, peg-140 glyceryl tristearate, peg-15 cocomonium chloride, peg-15 glyceryl isostearate, peg-15 tallow polyamine, peg-150 dibehenate, peg-150 dilaurate, peg-150 dioleate, peg-150 lanolin, peg-150 polyglyceryl-2 tristearate, peg-160 sorbitan triisostearate, peg-175 diisostearate, peg-175 distearate, peg-18 palmitate, peg-190 distearate, peg-192 apricot kernel glycerides, peg-2 castor oil, peg-2 cocomonium chloride, peg-2 diethylhexanoate, peg-2 dilaurate, peg-2 distearate, peg-2 laurate SE, peg-2 oleamine, peg-2 oleammonium chloride, peg-2 ricinoleate, peg-2 stearmonium chloride, peg-20 cocamide MEA, peg-20 dilaurate, peg-20 distearate, peg-20 glyceryl laurate, peg-20 glyceryl oleate, peg-20 glyceryl ricinoleate, peg-20 glyceryl stearate, peg-20 hydrogenated lanolin, peg-20 laurate, peg-20 methyl glucose distearate, peg-20 methyl glucose sesquistearate, peg-20 palmitate, peg-20 phytosterol, peg-20 sorbitan cocoate, peg-20 stearate, peg-20 tallowate, peg-200 castor oil, peg-200 glyceryl tallowate, peg-200 hydrogenated castor oil, peg-200 hydrogenated glyceryl palmate, peg-200 trihydroxystearin, peg-22/dodecyl glycol copolymer, peg-23 glyceryl laurate, peg-23 olivate, peg-25 castor oil, peg-25 glyceryl oleate, peg-25 glyceryl stearate, peg-28 glyceryl tallowate, peg-29 castor oil, peg-3 cocamide, peg-3 dioleate, peg-3 dipalmitate, peg-30 dipolyhydroxystearate, peg-30 glyceryl cocoate, peg-30 glyceryl laurate, peg-30 glyceryl oleate, peg-30 glyceryl stearate, peg-30 hydrogenated tallow amine, peg-30 stearate, peg-32 dilaurate, peg-32 distearate, peg-35 castor oil, peg-4 dicocoate, peg-4 diheptanoate, peg-4 dioleate, peg-4 distearate, peg-4 laurate, peg-4 oleamide, peg-4 olivate, peg-40 castor oil, peg-40 glyceryl cocoate, peg-40 hydrogenated castor oil, peg-40 hydrogenated tallow amine, peg-40 sorbitan lanolate, peg-40 sorbitan peroleate, peg-40 stearate, peg-5 cocamide, peg-5 cocoate, peg-5 ethylhexanoate, peg-5 glyceryl sesquioleate, peg-5 hydrogenated tallow amine, peg-5 isononanoate, peg-5 oleamide, peg-5 sorbitan isostearate, peg-5 tallow amide, peg-50 hydrogenated tallow amine, peg-50 shea butter, peg-50 stearate, peg-50 tallow amide, peg-6 caprylic/capric glycerides, peg-6 dilaurate, peg-6 distearate, peg-6 isolauryl thioether, peg-6 oleamide, peg-6 palmitate, peg-60 almond glycerides, peg-7 glyceryl cocoate, peg-7 oleamide, peg-7 olivate, peg-7 ricinoleate, peg-7 stearate, peg-75 dilaurate, peg-75 distearate, peg-75 lanolin oil, peg-75 sorbitan lanolate, peg-78 glyceryl cocoate, peg-8 beeswax, peg-8 cocoate, peg-8 dilaurate, peg-8 dioleate, peg-8 distearate, peg-8 ditallate, peg-8 glyceryl laurate, peg-8 hydrogenated tallow amine, peg-8 isolauryl thioether, peg-8 laurate, peg-8 propylene glycol cocoate, peg-8 stearate, peg-8 tallow amide, peg-80 glyceryl cocoate, peg-80 glyceryl tallowate, peg-9 cocoate, peg-9 distearate, peg-9 oleamide, peg-9 oliveate, peg-9 stearamide carboxylic acid, peg/poly(1,2-butanediol)-52/32 dimethyl ether, peg/ppg-2/16 copolymer, peg/ppg-3/30 copolymer, peg/ppg-5/21 copolymer, peg/ppg-6/39 copolymer, peg/ppg-6/67 copolymer, peg/ppg-7/21 copolymer, peg/ppg-7/22 copolymer, peg/ppg-7/91 copolymer, peg/ppg-8/30 copolymer, peg/ppg-8/35 copolymer, peg/ppg-10/2 copolymer, peg/ppg-10/30 copolymer, peg/ppg-10/47 copolymer, peg/ppg-11/16 copolymer, peg/ppg-11/21 copolymer, peg/ppg-12/136 copolymer, peg/ppg-12/23 copolymer, peg/ppg-12/4 copolymer, peg/ppg-12/9 copolymer, peg/ppg-13/30 copolymer, peg/ppg-13/67 copolymer, peg/ppg-18/14 copolymer, peg/ppg-18/163 copolymer, peg/ppg-18/34 copolymer, peg/ppg-18/51 copolymer, peg/ppg-19/30 copolymer, peg/ppg-20/54 copolymer, peg/ppg-21/15 copolymer, peg/ppg-21/39 copolymer, peg/ppg-21/47 copolymer, peg/ppg-21/67 copolymer, peg/ppg-21/7 copolymer, peg/ppg-22/39 copolymer, peg/ppg-24/35 copolymer, peg/ppg-30/160 copolymer, peg/ppg-31/54 copolymer, peg/ppg-38/54 copolymer, peg/ppg-46/16 copolymer, peg/ppg-52/35 copolymer, peg/ppg-62/39 copolymer, peg/ppg-97/39 copolymer, peg/ppg-98/67 copolymer, peg/ppg-10/2 diricinoleate, peg/ppg-10/2 propylheptyl ether, peg/ppg-10/3 oleyl ether dimethicone, peg/ppg-12/16 dimethicone, peg/ppg-12/18 dimethicone, peg/ppg-122/47 copolymer, peg/ppg-128/54 copolymer, bis-peg/ppg-14/14 dimethicone, peg/ppg-14/2 propylheptyl ether, peg/ppg-14/4 dimethicone, peg/ppg-15/15 acetate dimethicone, peg/ppg-15/15 allyl ether acetate, peg/ppg-15/5 dimethicone, peg/ppg-150/30 copolymer, bis-peg/ppg-16/16 peg/ppg-16/16 dimethicone, peg/ppg-16/2 dimethicone, peg/ppg-16/8 dimethicone, peg/ppg-17/18 dimethicone, peg/ppg-18/18 dimethicone, peg/ppg-18/18 isostearate, peg/ppg-18/18 laurate, peg/ppg-18/6 copolymer, bis-peg/ppg-18/6 dimethicone, peg/ppg-19/19 dimethicone, peg/ppg-20/15 dimethicone, peg/ppg-20/20 dimethicone, bis-peg/ppg-20/20 dimethicone, peg/ppg-20/22 methyl ether dimethicone, peg/ppg-20/29 dimethicone, bis-peg/ppg-20/5 peg/ppg-20/5 dimethicone, peg/ppg-20/6 dimethicone, peg/ppg-22/22 butyl ether dimethicone, peg/ppg-22/23 butyl ether dimethicone, peg/ppg-22/23 dimethicone, peg/ppg-22/24 dimethicone, peg/ppg-23/23 butyl ether dimethicone, peg/ppg-24/18 butyl ether dimethicone, peg/ppg-25/25 dimethicone, peg/ppg-27/27 dimethicone, peg/ppg-27/9 butyl ether dimethicone, peg/ppg-3/10 dimethicone, peg/ppg-30/10 dimethicone, peg/ppg-32/3 diricinoleate, peg/ppg-4/12 dimethicone, peg/ppg-4/2 propylheptyl ether, peg/ppg-40/2 propylheptyl ether, peg/ppg-5/3 trisiloxane, peg/ppg-6/11 dimethicone, peg/ppg-6/2 glyceryl cocoate, peg/ppg-6/2 propylheptyl ether, peg/ppg-7/2 propylheptyl ether, peg/ppg-8/13 diisostearate, peg/ppg-8/14 dimethicone, peg/ppg-8/2 propylheptyl ether, peg/ppg-8/3 diisostearate, peg/ppg-8/3 laurate, pentaerythrityl distearate, pentaerythrityl tetraisostearate, neopentyl glycol diisostearate, neopentyl glycol dilaurate, hydrogenated phosphatidylcholine, phosphatidylcholine, egg yolk, phosphatidylglycerol, Pinus sibirica seed oil polyglyceryl-6 esters, poloxamer 105 benzoate, poloxamer 182 dibenzoate, poloxamine 1101, poloxamine 1102, poloxamine 1104, poloxamine 1301, poloxamine 1302, poloxamine 1304, poloxamine 1307, poloxamine 1501, poloxamine 1502, poloxamine 1508, poloxamine 304, poloxamine 504, poloxamine 604, poloxamine 701, poloxamine 702, poloxamine 704, poloxamine 707, poloxamine 901, poloxamine 904, poloxamine 908, poly(C6-14 olefin), polyacrylate-29, polyester-6, polyglyceryl dimer soyate, polyglyceryl-2 oleate, polyglyceryl-2 stearate, polyglyceryl-4 oleate, polyglyceryl-4 pentastearate, polyglyceryl-4 stearate, polyglyceryl-6 caprylate, polyglyceryl-6 distearate, polyglyceryl-6 laurate, polyglyceryl-6 myristate, polyglyceryl-6 octastearate, polyglyceryl-6 oleate, polyglyceryl-6 pentastearate, polyglyceryl-6 polyricinoleate, polyglyceryl-6 tristearate, polyglyceryl-8 oleate, polyglyceryl-8 stearate, polyglyceryl-10 decamacadamiate, polyglyceryl-10 diisostearate, polyglyceryl-10 dioleate, polyglyceryl-10 distearate, polyglyceryl-10 pentaisostearate, polyglyceryl-10 pentaoleate, polyglyceryl-10 apricot kernelate, polyglyceryl-10 behenate/eicosadioate, polyglyceryl-10 bis(hydroxypropyl lauryl ether), polyglyceryl-10 caprate, polyglyceryl-10 caprylate, polyglyceryl-10 cocoate, polyglyceryl-10 decahydroxystearate, polyglyceryl-10 decalinoleate, polyglyceryl-10 dicocoate, polyglyceryl-10 didecanoate, polyglyceryl-10 dimyristate, polyglyceryl-10 dipalmitate, polyglyceryl-10 dodecabehenate, polyglyceryl-10 dodecacaprate, polyglyceryl-10 dodecacaprylate, polyglyceryl-10 hepta(behenate/stearate), polyglyceryl-10 hexaerucate, polyglyceryl-10 hexaisostearate, polyglyceryl-10 hexaoleate, polyglyceryl-10 hydroxypropyl ethylhexyl ether, polyglyceryl-10 hydroxypropyl lauryl ether, polyglyceryl-10 lauryl ether, polyglyceryl-10 palmate, polyglyceryl-10 pentacaprylate, polyglyceryl-10 pentalaurate, polyglyceryl-10 pentalinoleate, polyglyceryl-10 pentaricinoleate, polyglyceryl-10 polyhydroxystearate, polyglyceryl-10 sesquistearate, polyglyceryl-10 tricocoate, polyglyceryl-10 tridecanoate, polyglyceryl-12 bis(hydroxypropyl ethylhexyl ether), polyglyceryl-2 caprate, polyglyceryl-2 caprylate, polyglyceryl-2 diisostearate, polyglyceryl-2 distearate, polyglyceryl-2 hydroxypropyl ethylhexyl ether, polyglyceryl-2 hydroxypropyl lauryl ether, polyglyceryl-2 isononanoate/dimer dilinoleate copolymer, polyglyceryl-2 isopalmitate, polyglyceryl-2 isopalmitate/sebacate, polyglyceryl-2 lanolin alcohol ether, polyglyceryl-2 laurate, polyglyceryl-2 lauryl ether, polyglyceryl-2 myristate, polyglyceryl-2 oleyl ether, polyglyceryl-2 palmitate, polyglyceryl-2 sesquicaprylate, polyglyceryl-2 sesquioleate, polyglyceryl-2 sesquistearate, polyglyceryl-2 sorbitan tetraethylhexanoate, polyglyceryl-2 tetraoleate, polyglyceryl-2 tetrastearate, polyglyceryl-2-peg-4 stearate, polyglyceryl-20 docosabehenate/isostearate, polyglyceryl-20 docosabehenate/laurate, polyglyceryl-20 docosabehenate/oleate, polyglyceryl-20 heptadecabehenate/laurate, polyglyceryl-20 octadecabehenate/laurate, polyglyceryl-20 octaisononanoate, polyglyceryl-3 beeswax, polyglyceryl-3 caprate, polyglyceryl-3 caprylate, polyglyceryl-3 cetyl ether, polyglyceryl-3 cetyl ether stearate, polyglyceryl-3 cocoate, polyglyceryl-3 decyltetradecyl ether, polyglyceryl-3 dicaprate, polyglyceryl-3 dicitrate/stearate, polyglyceryl-3 dicocoate, polyglyceryl-3 dihydroxy stearate, polyglyceryl-3 dihydroxystearate, isopolyglyceryl-3 dimethicone, isopolyglyceryl-3 dimethiconol, polyglyceryl-3 dioleate, polyglyceryl-3 distearate, polyglyceryl-3 hydroxylauryl ether, polyglyceryl-3 hydroxypropyl ethylhexyl ether, polyglyceryl-3 hydroxypropyl lauryl ether, polyglyceryl-3 isostearate, polyglyceryl-3 laurate, polyglyceryl-3 methylglucose distearate, polyglyceryl-3 myristate, polyglyceryl-3 oleyl ether phosphate, polyglyceryl-3 palmitate, polyglyceryl-3 pentaricinoleate, polyglyceryl-3 polyricinoleate, polyglyceryl-3 rice branate, polyglyceryl-3 rice branate phosphate, polyglyceryl-3 ricinoleate, polyglyceryl-3 sorbityl linseedate, polyglyceryl-3 soyate/shea butterate, polyglyceryl-3 stearate, polyglyceryl-3 stearate SE, polyglyceryl-3 triisostearate, polyglyceryl-4 almondate/shea butterate, polyglyceryl-4 caprate, polyglyceryl-4 caprylate, polyglyceryl-4 cocoate, polyglyceryl-4 diisostearate/polyhydroxystearate/sebacate, polyglyceryl-4 dilaurate, polyglyceryl-4 distearate, polyglyceryl-4 hazelseedate, polyglyceryl-4 hydroxypropyl ethylhexyl ether, polyglyceryl-4 isostearate, polyglyceryl-4 isostearate/laurate, polyglyceryl-4 laurate, polyglyceryl-4 lauryl ether, polyglyceryl-4 oleyl ether, polyglyceryl-4 pentaoleate, polyglyceryl-4 pentapalmitate/stearate, polyglyceryl-4 polyricinoleate, polyglyceryl-4 sorbityl ether olivate phosphate, polyglyceryl-4 sweet almondate, polyglyceryl-4 tristearate, polyglyceryl-4-peg-2 cocamide, polyglyceryl-5 bis(hydroxypropyl ethylhexyl ether), polyglyceryl-5 caprate, polyglyceryl-5 dilaurate, polyglyceryl-5 dioleate, polyglyceryl-5 hexastearate, polyglyceryl-5 hydroxypropyl lauryl ether, polyglyceryl-5 isostearate, polyglyceryl-5 laurate, polyglyceryl-5 myristate, polyglyceryl-5 oleate, polyglyceryl-5 pentamyristate, polyglyceryl-5 polyricinoleate, polyglyceryl-5 stearate, polyglyceryl-5 tribehenate, polyglyceryl-5 triisostearate, polyglyceryl-5 trimyristate, polyglyceryl-5 trioleate, polyglyceryl-5 tristearate, polyglyceryl-6 Adansonia digitata seedate, polyglyceryl-6 apricot kernelate, polyglyceryl-6 argan kernelate, polyglyceryl-6 bis(hydroxypropyl ethylhexyl ether), polyglyceryl-6 bis(hydroxypropyl lauryl ether), polyglyceryl-6 caprate, polyglyceryl-6 Citrullus lanatus seedate, polyglyceryl-6 dicaprate, polyglyceryl-6 diisostearate, polyglyceryl-6 dioleate, polyglyceryl-6 dipalmitate, polyglyceryl-6 heptacaprylate, polyglyceryl-6 hexaoleate, polyglyceryl-6 hexastearate, polyglyceryl-6 isostearate, polyglyceryl-6 myristyl ether, polyglyceryl-6 palmitate, polyglyceryl-6 palmitate/succinate, polyglyceryl-6 pentaoleate, polyglyceryl-6 pentaricinoleate, polyglyceryl-6 polyhydroxystearate, polyglyceryl-6 ricinoleate, polyglyceryl-6 sesquiisostearate, polyglyceryl-6 sesquistearate, polyglyceryl-6 tetrabehenate, polyglyceryl-6 tetraoleate, polyglyceryl-6 undecylenate, polyglyceryl-8 C₁₂₋₂₀ acid ester, polyhydroxystearic acid, polyperfluoroethoxymethoxy peg-2 phosphate, polyquaternium-75, polyquaternium-77, polyquaternium-78, polyquatemium-79, polyquaternium-80, polyquaternium-81, polyquaternium-88, polysorbate 20, polysorbate 21, polysorbate 40, polysorbate 60, polysorbate 61, polysorbate 65, polysorbate 80, polysorbate 80 acetate, polysorbate 85, polyurethane-28, potassium babassuate, potassium cocoate, potassium cocoyl rice amino acids, potassium cornate, potassium deceth-4 phosphate, potassium dextrin octenylsuccinate, potassium dimethicone peg-7 phosphate, potassium laurate, potassium laureth phosphate, potassium lauryl sulfate, potassium myristate, potassium octoxynol-12 phosphate, potassium oleate, potassium olivate, potassium palmitate, potassium peanutate, potassium peg-50 hydrogenated castor oil succinate, potassium stearate, potassium tallowate, potassium tsubakiate, potassium undecylenate, ppg-1 beheneth-15, ppg-1 hydroxyethyl caprylamide, ppg-1 trideceth-13, ppg-1-deceth-4, ppg-1-deceth-5, ppg-1-deceth-6, ppg-1-deceth-7, ppg-1-isodeceth-4, ppg-1-isodeceth-6, ppg-1-isodeceth-7, ppg-1-isodeceth-9, ppg-1-peg-9 lauryl glycol ether, ppg-2 C₁₂₋₁₃ pareth-8, ppg-2 C₁₂₋₁₅ pareth-6, ppg-2 C9-11 pareth-5, ppg-2 C9-11 pareth-7, ppg-2 C9-11 pareth-8, ppg-2 glyceryl ether, ppg-2 hydroxyethyl cocamide, ppg-2 isoceteth-20 acetate, ppg-2 lanolin alcohol ether, ppg-2-ceteareth-9, ppg-2-deceth-10, ppg-2-deceth-12, ppg-2-deceth-15, ppg-2-deceth-20, ppg-2-deceth-3, ppg-2-deceth-30, ppg-2-deceth-40, ppg-2-deceth-5, ppg-2-deceth-50, ppg-2-deceth-60, ppg-2-deceth-7, ppg-2-deceth-8, ppg-2-isodeceth-10, ppg-2-isodeceth-12, ppg-2-isodeceth-18, ppg-2-isodeceth-25, ppg-2-isodeceth-4, ppg-2-isodeceth-6, ppg-2-isodeceth-8, ppg-2-isodeceth-9, ppg-2-laureth-12, ppg-2-laureth-5, ppg-2-laureth-8, ppg-2-peg-11 hydrogenated lauryl alcohol ether, ppg-2-peg-6 coconut oil esters, ppg-2/peg-8 cocoate, ppg-3 C₁₂₋₁₄ sec-pareth-7, ppg-3 glyceryl ether, ppg-3 hydroxyethyl soyamide, ppg-3-deceth-2 carboxylic acid, ppg-3-isosteareth-9, ppg-3-laureth-10, ppg-3-laureth-12, ppg-3-laureth-8, ppg-3-laureth-9, ppg-3-myreth-11, ppg-3-myreth-3, ppg-3-peg-6 oleyl ether, ppg-4 C₁₂₋₁₄ sec pareth-7, ppg-4 C₁₂₋₁₄ sec-pareth-5, ppg-4 C13-15 pareth-15, ppg-4 laureth-7, ppg-4 trideceth-6, ppg-4-ceteareth-12, ppg-4-ceteth-10, ppg-4-ceteth-20, ppg-4-deceth-4, ppg-4-deceth-6, ppg-4-isodeceth-10, ppg-5 C₁₂₋₁₄ sec-pareth-7, ppg-5 C₁₂₋₁₄ sec-pareth-9, ppg-5 C9-15 pareth-6, ppg-5 phytosterol, ppg-5-ceteth-20, ppg-6 C12-18 pareth-11, ppg-6 glyceryl ether, ppg-6 trideceth-8, ppg-6-deceth-9, ppg-6-decyltetradeceth-12, ppg-6-decyltetradeceth-20, ppg-6-decyltetradeceth-30, bis-(ppg-7 undeceneth-21) dimethicone, ppg-7/peg-30 phytosterol, ppg-8 glyceryl ether, ppg-8 polyglyceryl-2 ether, ppg-8-ceteth-20, ppg-8-deceth-6, ppg-9 glyceryl ether, ppg-9-ethylhexeth-5, ppg-10 glyceryl ether, ppg-10 jojoba alcohol, ppg-10 lanolin alcohol ether, ppg-10-ceteareth-20, ppg-12 capryleth-18, ppg-12 diglucosyl C14-18 acidate, ppg-12-laneth-50, ppg-12-peg-65 lanolin oil, ppg-13 decyltetradeceth-24, ppg-14 diglyceryl ether, ppg-14 polyglyceryl-2 ether, ppg-14-deceth-6, ppg-16 glyceryl ether, ppg-20 lanolin alcohol ether, ppg-20-decyltetradeceth-10, ppg-21 butyl ether phosphate, ppg-23-peg-4 trimethylolpropane, ppg-23-steareth-34, ppg-25 butyl ether phosphate, ppg-25-glycereth-22, ppg-27 glyceryl ether, ppg-30 ethylhexeth-4 phosphate, ppg-30 lanolin alcohol ether, ppg-30 steareth-4, ppg-34 steareth-3, ppg-35 butyl ether phosphate, ppg-40-peg-60 lanolin oil, ppg-50 glyceryl ether, ppg-10 cetyl ether phosphate, ppg-12-peg-50 lanolin, ppg-24-glycereth-24, ppg-25-laureth-25, ppg-5-ceteth-10 phosphate, ppg-5-laureth-5, ppg-6 C₁₂₋₁₅ pareth-12, ppg-6 C9-11 pareth-5, ppg-6-deceth-4, ppg-66-glycereth-12, ppg-9 diethylmonium chloride, ppg/peg-2/10 glyceryl cocoate, isopropyl lanolate, isopropyl titanium triisostearate, propylene glycol capreth-4, propylene glycol dicocoate, propylene glycol hyaluronate, propylene glycol isodeceth-4, propylene glycol oleate SE, propylene glycol oleth-5, propylene glycol ricinoleate, propylene glycol stearate, propylene glycol stearate SE, pumpkin seed oil peg-8 esters, quaternium-90 montmorillonite, quaternium-92, Quillaja saponaria bark, Quillaja saponaria bark extract, raffinose myristate, raffinose oleate, rapeseed glucoside, rapeseed glyceride, hydrogenated rapeseed glycerides, rapeseed oil peg-3 esters, rapeseed oil peg-20 esters, rapeseed oil sorbitol esters, raspberry seed oil peg-8 esters, rhamnolipids, rice bran oil polyglyceryl-3 esters, rice oil diglyceryl esters, (Z)-, ricinoleic acid, ricinoleth-40, Rosa rubiginosa seed oil peg-8 esters, Saccharomyces/Stevia rebaudiana leaf/stem ferment extract, safflower seed oil peg-8 esters, safflower seed oil polyglyceryl-6 esters, Schinziophyton rautanenii kernel oil peg-8 esters, Schinziophyton rautanenii kernel oil polyglyceryl-6 esters, Sclerocarya birrea seed oil peg-8 esters, Sclerocarya birrea seed oil polyglyceryl-6 esters, Sclerocarya birrea seed oil polyglyceryl-10 esters, sesame oil polyglyceryl-6 esters, sesame seed oil peg-8 esters, sesamide DIPA, rhea butter glycereth-8 esters, shea butter glyceride, shea butter peg-8 esters, shea butter peg-32 esters, shea butter polyglyceryl-6 esters, shea butteramidopropyltrimonium chloride, silicone quaternium-17, silicone quaternium-21, sodium avocadoate, sodium beeswax, sodium behenoyl lactylate, sodium bisglycol ricinosulfosuccinate, sodium butoxynol-12 sulfate, sodium butylglucosides hydroxypropyl phosphate, sodium C10-15 pareth sulfate, sodium C₁₁₋₁₅ pareth-7 carboxylate, sodium C₁₂₋₁₃ pareth sulfate, sodium C₁₂₋₁₃ pareth-12 carboxylate, sodium C₁₂₋₁₃ pareth-5 carboxylate, sodium C₁₂₋₁₃ pareth-8 carboxylate, sodium C₁₂₋₁₄ pareth-3 sulfate, sodium C₁₂₋₁₅ alkyl sulfate, sodium C₁₂₋₁₅ pareth sulfate, sodium C₁₂₋₁₅ pareth-15 sulfonate, sodium C₁₂₋₁₅ pareth-3 sulfonate, sodium C₁₂₋₁₅ pareth-6 carboxylate, sodium C₁₂₋₁₅ pareth-7 carboxylate, sodium C₁₂₋₁₅ pareth-7 sulfonate, sodium C₁₂₋₁₅ pareth-8 carboxylate, sodium C12-18 alkyl sulfate, sodium C13-15 pareth-8 butyl phosphate, sodium C13-15 pareth-8 phosphate, sodium C₁₄₋₁₅ pareth-8 carboxylate, sodium C16-20 alkyl sulfate, sodium C9-11 pareth-6 carboxylate, sodium caproyl lactylate, sodium caprylate, sodium capryleth-9 carboxylate, sodium capryloamphohydroxypropylsulfonate, sodium caprylyl sulfonate, sodium carboxydecyl peg-8 dimethicone, sodium carboxymethyl C₁₀₋₁₆ alkyl glucoside, sodium carboxymethyl chitosan lauramide, sodium castorate, sodium ceteth-13 carboxylate, sodium ceteth-4 phosphate, sodium cetyl sulfate, sodium chitosan cocamide hydroxypropylsulfate, sodium chitosan lauramide hydroxypropylsulfate, sodium coco sulfoacetate, sodium coco-glucoside tartrate, sodium coco/babassu sulfate, sodium coco/hydrogenated tallow sulfate, sodium cocoate, sodium cocoglucosides hydroxypropyl phosphate, sodium cocoglucosides hydroxypropylsulfonate, sodium cocosulfate, sodium cocoyl barley amino acids, sodium cocoyl lactylate, sodium cocoyl threoninate, sodium cocoyl/palmoyl/sunfloweroyl glutamate, sodium cocoyl/stearoyl (alanine/arginine/asparagine/aspartic acid/glutamic acid/glutamine/glycine/histidine/isole, sodium deceth sulfate, sodium deceth-2 carboxylate, sodium decylglucosides hydroxypropylsulfonate, sodium diceteareth-10 phosphate, sodium dilaureth-4 phosphate, sodium dilaureth-7 citrate, sodium dioleth-8 phosphate, sodium glyceryl oleate phosphate, sodium hydroxylauryldimonium ethyl phosphate, sodium hydroxypropylphosphate decylglucoside crosspolymer, sodium hydroxypropylphosphate laurylglucoside crosspolymer, sodium hydroxypropylsulfonate cocoglucoside crosspolymer, sodium hydroxypropylsulfonate decylglucoside crosspolymer, sodium hydroxypropylsulfonate laurylglucoside crosspolymer, sodium isostearate, sodium isosteareth-11 carboxylate, sodium isosteareth-6 carboxylate, sodium isostearoyl lactate, sodium isostearoyl lactylate, sodium lardate, sodium lauraminopropionate, sodium laurate, sodium laureth sulfate, sodium laureth-2 phosphate, sodium laureth-3 carboxylate, sodium laureth-13 carboxylate, sodium laureth-4 phosphate, sodium lauroyl methylaminopropionate, sodium lauryl phosphate, sodium lauryl sulfate, sodium laurylglucosides hydroxypropyl phosphate, sodium laurylglucosides hydroxypropylsulfonate, sodium linoleate, sodium macadamiaseedate, sodium mangoseedate, sodium methoxy ppg-2 acetate, sodium methyl isethionate, sodium methyltaurine cocoyl methyltaurate, sodium myreth sulfate, sodium myristate, sodium myristyl sulfate, sodium octoxynol-2 ethane sulfonate, sodium octoxynol-6 sulfate, sodium octoxynol-9 sulfate, sodium oleoyl lactylate, sodium oleth sulfate, sodium oleth-7 phosphate, sodium oleth-8 phosphate, sodium oleyl sulfate, sodium palmate, sodium peanutate, sodium peg-3 lauramide carboxylate, sodium peg-4 lauramide carboxylate, sodium peg-50 hydrogenated castor oil succinate, sodium peg-6 cocamide carboxylate, sodium peg-8 cocamide carboxylate, sodium propoxy ppg-2 acetate, sodium rapeseedate, sodium ricinoleamidopropyl PG-dimonium chloride phosphate, sodium sesameseedate, sodium soyate, sodium steareth-4 phosphate, sodium stearoamphohydroxypropylsulfonate, sodium stearoyl glutamate, sodium stearoyl lactylate, sodium stearyl dimethyl glycine, sodium stearyl phthalamate, sodium stearyl sulfate, sodium surfactin, sodium sweet almondate, sodium tallate, sodium taurine cocoyl methyltaurate, sodium trideceth sulfate, sodium tridecyl sulfate, sodium undeceth-5 carboxylate, sodium/MEA laureth-2 sulfosuccinate, sodium/MEA-peg-3 cocamide sulfate, sorbeth-2 beeswax, sorbeth-2 cocoate, sorbeth-3 isostearate, sorbeth-3 tristearate, sorbeth-4 tetraoleate, sorbeth-5, sorbeth-6 beeswax, sorbeth-6 hexastearate, sorbeth-6 laurate, sorbeth-6 tetraoleate, sorbeth-7, sorbeth-8 beeswax, sorbeth-20 pentaisostearate, sorbeth-20 tetraisostearate, sorbeth-30 pentaisostearate, sorbeth-30 tetraisostearate, sorbeth-30 tetraoleate laurate, sorbeth-40 pentaisostearate, sorbeth-40 pentaoleate, sorbeth-40 tetraisostearate, sorbeth-50 pentaisostearate, sorbeth-50 tetraisostearate, sorbeth-60 tetrastearate, sorbeth-20 beeswax, sorbeth-30, sorbeth-30 tetraoleate, sorbeth-40, sorbeth-40 tetraoleate, sorbeth-50 hexaoleate, sorbeth-6, sorbeth-60 tetraoleate, isosorbide laurate, sorbitan caprylate, sorbitan cocoate, sorbitan diisostearate, sorbitan dioleate, sorbitan isostearate, sorbitan laurate, sorbitan oleate, sorbitan oleate decylglucoside crosspolymer, sorbitan oleate laurylglucoside crosspolymer, sorbitan olivate, sorbitan palmitate, sorbitan sesquiisostearate, sorbitan sesquioleate (2:3), sorbitan sesquistearate, sorbitan stearate, sorbitan triisostearate, sorbitan trioleate, sorbitan tristearate, sorbitan undecylenate, sorbityl acetate, sorbityl laurate, soy acid, hydrogenated soy polyglycerides/soyamide DEA, soy sterol, soy sterol acetate, soyamide DEA, soyamidopropyl dimethylamine, soyamine, soyaminopropylamine, soybean oil peg-8 esters, soybean oil peg-20 esters, soybean oil peg-36 esters, soybean oil polyglyceryl-6 esters, soytrimonium chloride, stearamidoethyl ethanolamine, isostearamidomorpholine stearate, stearamidopropyl dimethylamine stearate, steardimonium hydroxypropyltrimonium chloride, steareth-1, steareth-2, isosteareth-2, steareth-2 phosphate, steareth-3, isosteareth-3, steareth-4, steareth-5, isosteareth-5, steareth-5 stearate, steareth-6, isosteareth-6 carboxylic acid, steareth-7, steareth-8, steareth-10, isosteareth-10, isosteareth-10 stearate, steareth-11, isosteareth-11 carboxylic acid, steareth-12, isosteareth-12, steareth-13, steareth-14, steareth-15, isosteareth-15, steareth-16, steareth-20, isosteareth-20, steareth-21, isosteareth-22, steareth-25, isosteareth-25, isosteareth-50, isosteareth-2 phosphate, steareth-200, isosteareth-200 palmitate, isostearic/myristic glycerides, stearoamphoacetic acid, stearoyl ethyltrimonium methosulfate, stearoyl inulin, stearoyl lactylic acid, stearoyl leucine, stearyl acetyl glutamate, stearyl alcohol, isostearyl carboxydecyl peg-8 dimethicone, isostearyl glucoside, stearyl olivate, stearyl PG-trimonium chloride, stearyl phosphate, stearyldimoniumhydroxypropyl butylglucosides chloride, stearyldimoniumhydroxypropyl decylglucosides chloride, stearyldimoniumhydroxypropyl laurylglucosides chloride, sucrose cocoate, sucrose dilaurate, sucrose distearate, sucrose hexaerucate, sucrose hexaoleate/hexapalmitate/hexastearate, sucrose hexapalmitate, sucrose mortierellate, sucrose myristate, sucrose oleate, sucrose palmitate, sucrose pentaerucate, sucrose polybehenate, sucrose polycottonseedate, sucrose polylaurate, sucrose polylinoleate, sucrose polypalmate, sucrose polysoyate, sucrose polystearate, sucrose ricinoleate, sucrose tetraisostearate, sucrose tetrastearate triacetate, sucrose tribehenate, sucrose trilaurate, sucrose tristearate, sunflower glycerides citrate, sunflower seed oil glycereth-8 esters, sunflower seed oil glyceride, sunflower seed oil glycerides, sunflower seed oil peg-32 esters, sunflower seed oil peg-8 esters, sunflower seed oil polyglyceryl-10 esters, sunflower seed oil polyglyceryl-3 esters, sunflower seed oil polyglyceryl-4 esters, sunflower seed oil polyglyceryl-5 esters, sunflower seed oil polyglyceryl-6 esters, sunflower seed oil sorbitol esters, hydrogenated tallow acid, tallow amine, acetylated hydrogenated tallow glyceride, hydrogenated tallow glyceride citrate, hydrogenated tallow glyceride lactate, tallow glycerides, acetylated hydrogenated tallow glycerides, hydrogenated tallow glycerides, hydrogenated tallow glycerides citrate, dihydrogenated tallow methylamine, dihydrogenated tallow phthalic acid amide, tallowaminopropylamine, tallowdimonium propyltrimonium dichloride, talloweth-5, talloweth-7, hydrogenated talloweth-12, talloweth-18, hydrogenated talloweth-25, hydrogenated talloweth-60 myristyl glycol, talloweth-4, talloweth-6, TEA-C₁₁₋₁₅ pareth sulfate, TEA-C₁₂₋₁₃ pareth-3 sulfate, TEA-canolate, TEA-cocoate, TEA-cocoyl glycinate, TEA-dextrin octenylsuccinate, TEA-diethanolaminoethyl polyisobutenylsuccinate, TEA-dimethicone peg-7 phosphate, TEA-isostearate, TEA-laneth-5 sulfate, TEA-laurate, TEA-laurate/myristate, TEA-laureth sulfate, TEA-laureth-4 phosphate, TEA-lauroyl lactylate, TEA-lauryl sulfate, TEA-myristate, TEA-oleate, TEA-oleyl sulfate, TEA-palmitate, TEA-peg-3 cocamide sulfate, TEA-peg-50 hydrogenated castor oil succinate, TEA-stearate, TEA-tallate, tetrasodium dicarboxyethyl stearyl sulfosuccinamate, Theobroma grandiflorum seed butter glyceryl esters, Theobroma grandiflorum seed butter peg-8 esters, Theobroma grandiflorum seed butter polyglyceryl-6 esters, TIPA-lauryl sulfate, TIPA-stearate, tocopheryl phosphate, tri-C₁₂₋₁₅ pareth-10 phosphate, tri-C₁₂₋₁₅ pareth-2 phosphate, tri-C₁₂₋₁₅ pareth-6 phosphate, tri-C₁₂₋₁₅ pareth-8 phosphate, triarachidin, tribehenin peg-20 esters, triceteareth-4 phosphate, triceteth-5 phosphate, Trichilia emetica seed oil peg-8 esters, Trichilia emetica seed oil polyglyceryl-6 esters, trideceth-2, trideceth-3, trideceth-3 phosphate, trideceth-4, trideceth-5, trideceth-6, trideceth-6 phosphate, trideceth-7, trideceth-8, trideceth-9, trideceth-10 phosphate, trideceth-12, trideceth-15, trideceth-15 carboxylic acid, trideceth-18, trideceth-20, trideceth-50, trifluoropropyl dimethicone/peg-10 crosspolymer, trilaneth-4 phosphate, trilaureth-4 phosphate, trilaureth-9 citrate, triolein peg-6 esters, trioleth-8 phosphate, undecanoic acid dimethicones, undeceth-3, undeceth-5, undeceth-7, undeceth-8, undeceth-9, undeceth-11, hydrolyzed VA/vinyl acetoacetate copolymer, hydrogenated vegetable glycerides, hydrogenated vegetable glycerides citrate, vegetable glycerides phosphate, hydrogenated vegetable glycerides phosphate, watermelon seed oil peg-8 esters, watermelon seed oil polyglyceryl-10 esters, watermelon seed oil polyglyceryl-6 esters, wheat germ oil glycereth-8 esters, wheat germ oil peg-8 esters, xanthan gum, ximenia americana seed oil peg-8 esters, ximenia americana seed oil polyglyceryl-6 esters, zinc coceth sulfate, zinc coco-sulfate, and mixtures thereof.

In some embodiments, the present invention provides compositions comprising: from about 50 wt. % to about 60 wt. % dimethicone polysilicone-11, from about 3 wt. % to about 20 wt. % caprylyl methicone, from about 2 wt. % to about 12 wt. % dimethicone (and) dimethicone/vinyl dimethicone crosspolymer, from about 1 wt. % to about 7 wt. % dimethyl isosorbide, 1 wt % to about 10 wt. % caprylic/capric triglyceride, from about 1 wt. % to about 5 wt. % of an oil w/ <50% polyunsaturated fatty acids (e.g. Persea gratissima oil), from about 1 wt. % to about 4 wt. % PPG-12/SMD1 copolymer, from about 1 wt. % to about 6 wt. % ethoxydiglycol, from about 0.5 wt. %© to about 5 wt. % Cannabis sativa (hemp) seed oil, from about 0.1 wt. % to about 5 wt. % palmitoylethanolamide, from about 0.1 wt. % to about 5 wt. % Cannabis sativa (hemp) oil, from about 0.05 wt. % to about 4 wt. %, from about 0.1 to about 5 wt. % oleic acid, from about 0.5 to about 3 wt. % cetyl PEG/PPG-10/1 dimethicone, from about 0.25 wt. % to about 5 wt. % Avena sativa (oat) kernel flour (colloidal oatmeal) and from about 0.05 wt. % to about 2 wt. % bisabolol. In some embodiments, the colloidal oatmeal is in the form of an oil, rather than a dry form (e.g. flour or powder).

In some embodiments, the present invention provides personal care compositions comprising a first hydrophobic material comprising a semi-solid mixture of hydrocarbons; a second hydrophobic material comprising a complex comprising a semi-solid mixture of hydrocarbons and a microcrystalline cellulose wax; a cannabinoid; a terpene; and a cosmetically acceptable carrier. In other embodiments, the semi-solid mixture of hydrocarbons comprises petrolatum or a wax having a similar consistency. In further embodiments, the first hydrophobic material is present in an amount of from about 5 wt. % to about 15 wt. % of the composition. In some embodiments, the first hydrophobic material is present in an amount of from about 7.5 wt. % to about 12.5 wt. % of the composition. In other embodiments, the first hydrophobic material is present in an amount of from about 10 wt. % to about 12 wt. % of the composition. Still further embodiments provide compositions wherein the first hydrophobic material is present in an amount of about 11 wt. % of the composition.

Other embodiments provide compositions wherein the semi-solid mixture of hydrocarbons in the complex comprising a semi-solid mixture of hydrocarbons and a microcrystalline cellulose wax comprises petrolatum or a wax having a similar consistency. In some embodiments, the second hydrophobic material is present in an amount of from about 35 wt. % to about 45 wt. % of the composition. In some embodiments, the second hydrophobic material is present in an amount of from about 36 wt. % to about 44 wt. % of the composition. In some embodiments, the second hydrophobic material is present in an amount of from about 37 wt. % to about 43 wt. % of the composition. In some embodiments, the second hydrophobic material is present in an amount of from about 38 wt. % to about 42 wt. % of the composition. In some embodiments, the second hydrophobic material is present in an amount of from about 39 wt. % to about 41 wt. % of the composition. In some embodiments, the second hydrophobic material is present in an amount of about 40 wt. % of the composition.

Still further embodiments provide personal care compositions wherein the first hydrophobic material and the second hydrophobic material are present in a weight ratio range of from about 1:2 to about 1:5. While other embodiments provide personal care compositions wherein the first hydrophobic material and the second hydrophobic material are present in a weight ratio range of from about 1:2.5 to about 1:5. Yet other embodiments provide personal care compositions wherein the first hydrophobic material and the second hydrophobic material are present in a weight ratio range of from about 1:3 to about 1:5. In some embodiments, the first hydrophobic material and the second hydrophobic material are present in a weight ratio range of about 1:4.

In some embodiments, the present invention provides personal care compositions comprising a first hydrophobic material comprising a semi-solid mixture of hydrocarbons; a second hydrophobic material comprising a complex comprising a semi-solid mixture of hydrocarbons and a microcrystalline cellulose wax; a cannabinoid; a terpene; Avena sativa kernel flour; and a cosmetically acceptable carrier.

In some embodiments, the compositions described herein comprise a thickener. Examples of thickners include agar, algin (Laminaria spp. and other kelps), alginic acid, alumina magnesium metasilicate, ammonium alginate, astragalus gummifer gum, attapulgite, biotite, Caesalpinia spinosa gum, calcium alginate, calcium carrageenan, carrageenan, cassia gum, cellulose, cellulose regenerated, Cyamopsis tetragonoloba gum, dextrin, ethyl hydroxyethyl cellulose, gellan gum, ghatti gum, hydrogenated rapeseed oil, lithium magnesium silicate, lithium magnesium sodium silicate, methyl ethyl cellulose, pectin, potassium alginate, potassium carrageenan, propylene glycol alginate, sodium carboxymethyl cellulose, sodium carrageenan, sodium hydroxypropyl starch phosphate, sodium magnesium silicate, sodium sulfate, Sterculia urens gum, synthetic fluorphlogopite, vinyl dimethyl/trimethylsiloxysilicate stearyl dimethic one crosspolymer, vinyldimethyl/trimethylsiloxysilicate stearyl dimethicone crosspolymer, xanthan gum, and mixtures thereof.

In further embodiments, the compositions described herein comprise a fragrance. Examples of fragrances that may be used include abronia fragrance, acacia fragrance, agate fragrance, aldehydic fragrance, allspice fragrance, almond fragrance, aloe fragrance, aloe vera fragrance, amaretto coffee fragrance, amber floral fragrance, amber fragrance, sports amber fragrance, amber nutmeg fragrance, amber powdery floral fragrance, amber romance fragrance, ambergris fragrance, ambreine fragrance, ambrosia fragrance, angel dust fragrance, angel essence fragrance, angel feathers fragrance, angel wings fragrance, animal fragrance, anise fragrance, apple blossom fragrance, apple butter fragrance, apple cider fragrance, apple cinnamon fragrance, apple cranberry fragrance, apple crisp fragrance, apple fragrance, baked apple fragrance, candy apple fragrance, green apple fragrance, red apple fragrance, apple pear fragrance, apple pie fragrance, apple spice fragrance, apricot almond fragrance, apricot fragrance, apricot mango fragrance, apricot peach fragrance, aromatic fragrance, aromatic woody citrus floral fragrance, autumn fragrance, avocado fragrance, azalea fragrance, baby powder fragrance, bacon fragrance, balsam fragrance, bamboo fragrance, banana cream pie fragrance, banana fragrance, banana nut bread fragrance, banana nut cake fragrance, banana pudding fragrance, bay rum fragrance, bayberry fragrance, bergamot blossom fragrance, bergamot fragrance, berry fragrance, china berry fragrance, wild berry fragrance, berry jam fragrance, bird of paradise fragrance, blackberry fragrance, blackberry vanilla fragrance, blue grass fragrance, blue lagoon fragrance, blueberry fragrance, blueberry muffin fragrance, blueberry pie fragrance, boil de rose fragrance, boronia fragrance, bouquet fragrance, bouvardia fragrance, boysenberry fragrance, brazil nut fragrance, baked bread fragrance, briar wood fragrance, bubble gum fragrance, butter cream fragrance, butter fragrance, butter rum fragrance, butter vanilla fragrance, butterscotch fragrance, cactus blossom fragrance, cake fragrance, pound cake fragrance, red velvet cake fragrance, spice cake fragrance, camellia fragrance, cananga fragrance, candle fragrance, candy cane fragrance, candy corn fragrance, cantaloupe fragrance, cappuccino fragrance, caramel corn fragrance, caramel fragrance, carnation fragrance, carrot cake fragrance, cashmere fragrance, cassia fragrance, castoreum fragrance, cedar forest fragrance, cedar fragrance, cedarwood fragrance, chamomile fragrance, champaca fragrance, cherry berry fragrance, cherry blossom fragrance, cherry blossom soap fragrance, cherry fragrance, black cherry fragrance, sour cherry fragrance, wild cherry fragrance, roasted chestnut fragrance, chocolate cherry fragrance, chocolate chip cookie fragrance, chocolate fragrance, dark chocolate fragrance, white chocolate fragrance, chocolate hazelnut fragrance, chocolate mint fragrance, chocolate orange fragrance, chocolate raspberry fragrance, christmas fragrance, chrysanthemum fragrance, chypre fragrance, cinnamon bun fragrance, cinnamon cookie fragrance, cinnamon fragrance, cinnamon spice fragrance, citronella fragrance, citrus basil fragrance, citrus bergamot fragrance, citrus floral fragrance, citrus floral green fragrance, citrus fragrance, citrus ginger fragrance, citrus green fragrance, citrus herbal fragrance, citrus linen fragrance, citrus mint fragrance, citrus rose fragrance, citrus sage fragrance, citrus vanilla fragrance, citrus woody floral fragrance, citrus woody fragrance, civet fragrance, clean linen fragrance, clematis fragrance, clove fragrance, clover fragrance, cocoa butter fragrance, cocoa fragrance, coconut cream pie fragrance, coconut fragrance, tropical coconut fragrance, coconut kiwi fragrance, coconut lime fragrance, coconut lime verbena fragrance, coconut mango fragrance, coconut papaya fragrance, coffee fragrance, cognac fragrance, cologne fragrance, colonia fragrance, cookie dough fragrance, cornucopia fragrance, coronilla fragrance, cotton candy fragrance, clean cotton fragrance, sea cotton fragrance, country meadow fragrance, crabapple blossom fragrance, cranberry fragrance, cranberry orange spice fragrance, cranberry spice fragrance, cream fragrance, creme brulee fragrance, cucumber fragrance, cucumber melon fragrance, cucumber rose melon fragrance, currant fragrance, black currant fragrance, red currant fragrance, cyclamen fragrance, daffodil fragrance, daisy fragrance, daphne fragrance, date fragrance, deodorant fragrance, dianthus fragrance, dillenia fragrance, dogwood fragrance, dragons blood fragrance, earth fragrance, egg nog fragrance, eglantine fragrance, elder berry fragrance, elder flower fragrance, evergreen fragrance, fagonia fragrance, fennel fragrance, fern fragrance, fetes fragrance, fig fragrance, filbert fragrance, fine fragrance, fir balsam fragrance, fir tree fragrance, floral aldehydic fragrance, floral bouquet fragrance, floral fragrance, citrus floral fragrance, fruity floral fragrance, green floral fragrance, spicy floral fragrance, woody floral fragrance, floral fruity fragrance, floral green fragrance, floral musk fragrance, floral oriental fragrance, floral powdery fragrance, floral spice fragrance, floral woody fragrance, flower shop fragrance, forest fragrance, forget me not fragrance, fougere herbal fragrance, fougere woody fragrance, frankincense fragrance, freesia fragrance, fresh & clean fragrance, fruit cocktail fragrance, fruit fragrance, fruity green fragrance, fungus fragrance, gardenia fragrance, gardenia lily fragrance, genet fragrance, geranium fragrance, ginger fragrance, white ginger fragrance, ginger lily fragrance, ginger peach fragrance, ginger spice oatmeal fragrance, ginger vanilla fragrance, gingerbread fragrance, gooseberry fragrance, gourmand fragrance, graham cracker fragrance, grape fragrance, concord grape fragrance, grapefruit fragrance, grapefruit ginger fragrance, grapefruit mango fragrance, grapefruit sage fragrance, green grass fragrance, sweet grass fragrance, green fragrance, spring green fragrance, green herbal fragrance, green leaf fragrance, greenhouse fragrance, grenadine fragrance, guava fragrance, guava tangerine fragrance, habuba fragrance, hair care fragrance, hawthorn fragrance, hazelnut coffee fragrance, hazelnut cream fragrance, hazelnut fragrance, heather fragrance, heliotrope fragrance, herbal fragrance, hibiscus fragrance, hollyberry fragrance, home care fragrance, honey almond fragrance, honey fragrance, honey rose fragrance, honeydew fragrance, honeysuckle fragrance, hop fragrance, huckleberry fragrance, hyacinth fragrance, hydrangea fragrance, incense fragrance, iris blossom fragrance, ivy fragrance, jasmin carnation fragrance, jasmin fragrance, jasmin muguet fragrance, jasmin vanilla fragrance, jonquil fragrance, juicy fragrance, juicy fruit fragrance, juniper berry fragrance, juniper fragrance, juniper sage fragrance, kewda fragrance, keylime pie fragrance, kiwi coconut fragrance, kiwi fragrance, kiwi strawberry fragrance, kumquat fragrance, lavandin fragrance, lavender blossom fragrance, lavender fougere fragrance, lavender fragrance, lavender vanilla fragrance, leather fragrance, Russian leather fragrance, leather woody fragrance, lemon balm fragrance, lemon bergamot fragrance, lemon blossom fragrance, lemon chiffon fragrance, lemon cucumber fragrance, lemon fragrance, lemon lime fragrance, lemon pie fragrance, lemon thyme fragrance, raspberry lemonade fragrance, lemongrass fragrance, licorice fragrance, lilac fragrance, lily fragrance, calla lily fragrance, water lily fragrance, lily of the valley fragrance, key lime cream fragrance, lime fragrance, lime sherbet fragrance, linden blossom fragrance, loganberry fragrance, lotus fragrance, lychee fragrance, mace fragrance, magnolia fragrance, maja fragrance, malt fragrance, mandarin fragrance, clementine mandarin fragrance, mandarin lemon fagrance, mandarin rose fragrance, mango citrus fragrance, mango flower fragrance, mango fragrance, mango mandarin fragrance, mango melon fragrance, mango peach fragrance, maple fragrance, lime margarita fragrance, marine fragrance, marzipan fragrance, meadowsweet fragrance, medicated fragrance, melon fragrance, citrus melon fragrance, midnight passion fragrance, milk fragrance, mimosa fragrance, mint fragrance, mistletoe fragrance, mocha fragrance, mock orange fragrance, morning glory fragrance, moss fragrance, moss rose fragrance, mulberry fragrance, mulberry spice fragrance, muscadine fragrance, musk fragrance, china musk fragrance, citrus musk fragrance, dark musk fragrance, egyptian musk fragrance, floral musk fragrance, fruity musk fragrance, jasmin musk fragrance, light musk fragrance, oriental musk fragrance, woody musk fragrance, myrrh fragrance, narcissus fragrance, nectar fragrance, neroli fragrance, new mown hay fragrance, nutmeg fragrance, oatmeal milk & honey fragrance, oatmeal raisin cookie fragrance, orange blossom fragrance, orange chiffon fragrance, orange cinnamon fragrance, orange clove fragrance, orange cranberry fragrance, orange cream fragrance, orange fragrance, orange ginger fragrance, orange marmalade fragrance, orange sherbet fragrance, orange spice fragrance, orange vanilla fragrance, orchid fragrance, orchid magnolia fragrance, oriental aldehydic fragrance, oriental citrus fragrance, oriental fragrance, oriental powdery fragrance, orris fragrance, osmanthus fragrance, fresh outdoors fragrance, pansy fragrance, papaya fragrance, papaya mango fragrance, papaya saffron fragrance, parijat fragrance, passion blossom fragrance, passion fruit fragrance, patchouli fragrance, patchouli woody amber fragrance, peach blossom fragrance, peach fragrance, peach pecan fragrance, peaches and cream fragrance, peanut butter cookie fragrance, peanut fragrance, pear berry fragrance, pear blossom fragrance, pear fragrance, pear spice fragrance, pecan pie fragrance, peony fragrance, peppermint fragrance, personal wash fragrance, petitgrain fragrance, petunia fragrance, phlox fragrance, pina colada fragrance, pine bouquet fragrance, pine forest fragrance, pine fragrance, christmas pine fragrance, pineapp+le fragrance, pinion fragrance, pistachio fragrance, pizza fragrance, plum fragrance, mirabelle plum fragrance, plumeria fragrance, pointsettia fragrance, pomegranate fragrance, popcorn fragrance, red poppy fragrance, potpourri fragrance, powder fragrance, powdery animal fragrance, pralines and cream fragrance, primrose fragrance, prune fragrance, pumpkin fragrance, pumpkin pie fragrance, pumpkin spice fragrance, quince fragrance, quince rum fragrance, rain forest fragrance, rain fragrance, china rain fragrance, spring rain fragrance, raisin fragrance, raspberry fragrance, black raspberry fragrance, reseda fragrance, rhubarb fragrance, rice flower & shea fragrance, root beer fragrance, rose d′orient fragrance, rose fragrance, desert rose fragrance, dog rose fragrance, red rose fragrance, tea rose fragrance, white rose fragrance, rose geranium fragrance, rose jasmin fragrance, rose musk fragrance, rose otto fragrance, rose petal fragrance, rose raspberry fragrance, rose woody fragrance, rosemary fragrance, rosemary mint fragrance, rum fragrance, saffron fragrance, sage fragrance, desert sage fragrance, sandalwood fragrance, vanilla sandalwood fragrance, sassafras fragrance, scented stock fragrance, sea breeze fragrance, Shalimar fragrance, sherry fragrance, skin care fragrance, smoke fragrance, soapy sandalwood moss fragrance, spearmint fragrance, spice fragrance, christmas spice fragrance, country spice fragrance, vanilla spice fragrance, spicy kitchen fragrance, spring flowers fragrance, spruce fragrance, stephanotis fragrance, strawberries and cream fragrance, strawberry fragrance, strawberry peach fragrance, sugar cookie fragrance, sugar fragrance, brown sugar fragrance, sunflower fragrance, sweet pea fragrance, tangerine fragrance, tangerine ginger fragrance, white tea and ginger fragrance, tea fragrance, black tea fragrance, chai tea fragrance, green tea fragrance, lemon tea fragrance, orange pekoe tea fragrance, white tea fragrance, teak fragrance, tobacco flower fragrance, tobacco fragrance, toffee fragrance, toilet waters, tomato leaf fragrance, tuberose fragrance, tulip fragrance, tutti frutti fragrance, vanilla fragrance, vanilla bean fragrance, vanilla cream fragrance, vanilla fragrance, country vanilla fragrance, french vanilla fragrance, vanilla hazelnut fragrance, verbena fragrance, vetiver fragrance, violet fragrance, violet leaf fragrance, violet shea fragrance, wallflower fragrance, walnut fragrance, watermelon fragrance, bourbon whiskey fragrance, wine fragrance, wintergreen fragrance, wisteria fragrance, witch hazel fragrance, and mixtures thereof.

Other embodiments provide a method of treating, preventing or ameliorating a symptom associated with a disease, disorder, or condition of the skin, comprising applying any one of the personal care compositions described herein, to the skin of a subject in need thereof. Still further embodiments provide the use of any one of the compositions described herein for treating, preventing or ameliorating a symptom associated with a disease, disorder, or condition of the skin. In some embodiments, the disease, disorder, or condition of the skin is selected from acne, dermatitis (e.g. atopic dermatitis, contact dermatitis, seborrheic dermatitis, or stasis dermatitis), eczema (e.g. dyshidrotic eczema, hand eczema or nummular eczema), neruodermatitis, lichen simplex chronicus, dry skin, baby rash, psoriasis, pruritis (e.g. neuropathic pruritis), burns, itch associated with burns, flaky skin, inflammation, keloids, epidermolysis bullosa, and the like.

Other embodiments provide a method of treating, preventing or ameliorating a symptom associated with a disease, disorder, or condition relating to both chronic or acute pain, comprising applying any one of the personal care compositions described herein, to the skin of a subject in need thereof. Still further embodiments provide the use of any one of the compositions described herein for treating, preventing or ameliorating a symptom associated with a disease, disorder, or condition relating to both chronic or acute pain. In some embodiments, the pain related disease, disorder, or condition is selected from neuropathic pain, including but not limited to postherpetic neuralgia, carpal tunnel syndrome, sciatica, and diabetic neuropathy. In some embodiments, the pain related disease, disorder, or condition is selected from joint pain, including but not limited to bursitis, tendinitis, rheumatoid arthritis, and osteoarthritis. In further embodiments, the pain related disease, disorder, or condition is selected from chronic back pain, including but not limited to discogenic pain, slipped or bulging discs, spinal stenosis, compression fractures, post-surgical recovery, scoliosis, or lordosis.

Further embodiments provide methods of using any one of the compositions described herein for treating or preventing pain associated with a disease, disorder or condition selected from: arthritis (e.g. osteoarthritis, psoriatic arthritis or rheumatoid arthritis); bursitis; carpal tunnel syndrome; central pain syndrome; cervical stenosis; complex regional pain syndrome; degenerative disc disease; peripheral neuropathy (e.g. diabetic peripherial neuropathy); Ehlers-Danlos syndrome; failed back surgery syndrome; fibromyalgia; headache; a herniated disc; a knee injury; migraine; myositis; pinched nerve; sciatica; spinal cord injury; spinal stenosis; TMJ, trigeminal neuralgia; whiplash; post herniorraphy pain syndrome; post mastectomy pain syndrome; post thorocotomy pain syndrome; trigger points; lupus; lyme disease; ulcerative colitis; scoliosis; spasmodic torticollis; Scheuemann's kyphosis disease; sacroiliac joint dysfunction; radiculopathy; postherpetic neuralgia (shingles); polymyositis; multiple sclerosis; juvenile dermatomyositis; irritable bowel sydrome; intercostal neuralgia; Guillain Barre syndrome; gout; glossopharyngeal neuralgia; eosinophilia-myalgia syndrome; endometriosis; dystonia; dermatomyositis; Crohn's disease; Ankylosing Spondylitis; Arachnoiditis; Arthrofibrosis; Avascular Necrosis; Burning Mouth Syndrome; Cadasil; Cauda Equina Syndrome; Charcot-Marie-Tooth (CMT) Disease; Chronic Fatigue Syndrome; and cerebral palsy.

In other embodiments, the pain is selected from: AIDS-related pain; back pain; cancer pain; growing pains; knee pain; leg pain; myofascial pain; neck pain; muscular pain; exercised induced inflammatory pain; neuropathic pain; pelvic pain; phantom limb pain; post stroke pain; and vascular pain.

Embodiments of the present invention will now be further described by way of the following, non-limiting, examples.

### EXAMPLES

### Example 1

Described in Table 1 (below) is an exemplary composition of the present invention.

TABLE 1

| | Weight |
|---|---|
| Ingredient | (w/w %) |
| Hydrophobic component | 80-98 |
| PPAR-α agonist(s) | 0.1-5 |
| Cannabinoid(s) | 0.1-10 |
| Oil w/>50% polyunsaturated fatty acids | 0.1-5 |
| (e.g. hemp seed oil) | |
| Oil w/<50% polyunsaturated fatty acids | 0.1-5 |
| Terpene(s) | 0.1-20 |
| Flavonoid(s) | 0-10 |
| Surfactant(s) | 0.1-5 |

### Example 2

The viscosity of an exemplary composition of the present invention comprising a hydrophobic component comprising a silicone crosspolymer and a similarly formulated comparative composition without a silicone crosspolymer are compared. It was discovered that the comparative composition, which did not contain the silicone crosspolymer, does not provide a consumer acceptable viscosity.

### Example 3

To determine the optimal concentration of silicone crosspolymer, various concentrations of silicone crosspolymer were tested in substantially similar compositions. It was discovered that an exemplary composition comprising, inter alia, silicone crosspolymer at a concentration of about 15 wt. %, performed better than a comparative composition comprising silicone crosspolymer at a concentration of about 10 wt. %.

### Example 4

To determine the optimal make-up of the hydrophobic component, the weight ratio of polydimethylsiloxane to silicone crosspolymer was evaluated. It was discovered that a composition comprising a weight ratio of polydimethylsiloxane:silicone crosspolymer of about 4:1, was superior to a composition comprising a weight ratio of polydimethylsiloxane:silicone crosspolymer of about 7:1.

### Example 5

Described in Table 2 (below) is an exemplary oil-in-water based composition according to some embodiments of the present invention.

TABLE 2

| | Weight |
|---|---|
| Ingredient | (w/w %) |
| Humectants (e.g. butylene glycol, glycerin) | 1-15 |
| Acrylic polymer | 0.1-5 |
| Surfactant (e.g. decyl glucoside) | 0.1-5 |
| Plasticizer (e.g. polysorbate) | 0.1-5 |
| pH adjusting agent (e.g. triethanolamine) | 0.1-5 |
| Preservative | 0.01-1 |
| Petrolatum | 1-20 |
| Oil w/<50% polyunsaturated fatty acids | 0.1-0.5 |
| Hydrophobic component (e.g. silicones) | 1-30 |
| Emollient (e.g. cetyl alcohol) | 0.1-5 |
| Terpenes | 0.1-5 |
| PPAR-α agonist(s) | 0.1-5 |
| Cannabinoid(s) | 0.1-10 |
| Water | 60-70 |

### Example 6

Described in Table 3 (below) is an exemplary lotion according to some embodiments of the present invention.

TABLE 3

| | Weight |
|---|---|
| Ingredient | (w/w %) |
| Humectants (e.g. butylene glycol, glycerin) | 1-15 |
| Acrylic polymer | 0.1-5 |
| Surfactant (e.g. decyl glucoside) | 0.1-5 |
| Plasticizer (e.g. polysorbate) | 0.1-5 |
| pH adjusting agent (e.g. sodium hydroxide) | 0.1-5 |
| Preservative | 0.01-1 |
| Petrolatum | 1-20 |
| Oil w/>50% polyunsaturated fatty acids | 0.1-0.5 |
| (e.g. hemp seed oil) | |
| Hydrophobic component (e.g. silicones) | 1-30 |
| Emollient (e.g. cetyl alcohol) | 0.1-5 |
| Terpenes | 0.1-5 |
| PPAR-α agonist(s) | 0.1-5 |
| Cannabinoid(s) | 0.1-10 |
| Water | 60-70 |

### Example 7

Described in Table 4 (below) is an exemplary salve according to certain embodiments of the present invention.

TABLE 4

| | Weight |
|---|---|
| Ingredient | (w/w %) |
| Hydrophilic wax (e.g. cera bellina) | 1-20 |
| Oil w/>50% polyunsaturated fatty acids | 20-60 |
| (e.g. hemp seed oil) | |
| Oil w/<50% polyunsaturated fatty acids | 20-60 |
| Terpenes | 0.1-5 |
| PPAR-α agonist(s) | 0.1-5 |
| Cannabinoid(s) | 0.1-10 |
| Fragrance/Essential Oils | 0.01-1 |

### Example 8

Described in Table 5 (below) is an exemplary composition according to certain embodiments of the present invention.

TABLE 5

| | Weight |
|---|---|
| Ingredient | (w/w %) |
| Hydrophobic Component (e.g. combination | 70-90% |
| of non-cyclic silicone components) | |
| Penetration enhancer(s) | 0-15% |
| Emollient(s) (e.g. long chain omega | 0-5% |
| 3/6/9 fatty acids) | |
| Emulsifier(s) or surfactant | 0.1-5% |
| Additional bioactive agent(s) | 0-5% |
| Cannabinoid(s) | 0.1-10% |
| PPAR-α agonist | 0.1-5% |
| Terpene(s) | 0-20% |
| Solubilizer(s) | 0-5% |

The compositions described in the foregoing Examples, can be prepared by conventional methods known to those skilled in the art.

### Example 9

Fifty-seven (57) healthy patients participated in a single blind, single center, study to investigate the irritation and sensitization potential of an exemplary composition of the present invention (e.g., a composition described above in Table 5) following repeated cutaneous patch applications. An exemplary composition of the preset invention comprising in relevant part a cannabinoid, a PPAR-α agonist; and a penetration enhancing system, was patched under occlusive conditions using Finn chambers. A total of nine induction patches were worn for forty-seven (47) hours (patching occurred Mondays, Wednesdays and Fridays) for three weeks (a make-up day was allowed to ensure subjects had all nine (9) induction patches). Subjects had a rest period of fourteen (14) days. Challenge patches were applied for forty-eight (48) hours and readings were made at one (1) hour and forty-eight (48) hours post removal. Four (4) subjects withdrew for personal reasons. However, no adverse events or reactions were reported. The exemplary composition of the present invention surprisingly met the standards required to substantiate claims for: “Dermatologically Tested”, “Clinically Tested”, “Clinically Proven”, “Kind to Skin”, “Mild for skin”, “Safe for Skin” and “Dermatologist Approved”. These results were even more remarkable, because the exemplary composition of the present invention did not include a cyclic silicone; and achieving the desired product profile in the absence of a cyclic silicone was not an easy challenge to overcome. Without being bound by theory, the present inventors believe that the surprising results observed in this study were attributable at least in part to the inventive penetration enhancing system of the present invention.

### Example 10

Various compositions were evaluated to identify compositions having the desired skin feel and consistency. The results of these evaluations illustrate that an exemplary composition of the present invention comprising in relevant part a first hydrophobic material comprising a semi-solid mixture of hydrocarbons; and a second hydrophobic material comprising a complex comprising a semi-solid mixture of hydrocarbons and a microcrystalline cellulose wax, wherein the weight ratio of the first hydrophobic material to the second hydrophobic material is about 1:4, provided the desired skin feel and consistency, while several comparative compositions, which did not include the inventive combination of ingredients, failed to provide the desired skin feel or consistency. These results were truly unexpected given that all of the compositions that were evaluated included approximately the same total amount of hydrophobic material. The differences involved inter alia—the type of hydrophobic material(s) and/or the weight ratio of the first and second hydrophobic materials.

### Example 11

Table 6 below describes the formulas of several exemplary compositions of the present invention.

TABLE 6

| | 1 | 2 | 3 | 4 | 5 | 6 | 7 | 8 |
|---|---|---|---|---|---|---|---|---|
| Ingredient | Wt. % | | | | | | | |
| Petrolatum | — | 25 | 51 | 11 | 44 | — | — | — |
| Petrolatum + Microcrystalline | 51 | 25 | — | 40 | — | — | — | — |
| Cellulose Wax | | | | | | | | |
| Microcrystalline Cellulose Wax | — | — | — | — | 7 | — | — | — |
| Luteolin | — | — | — | — | — | 1 | 1 | — |
| Non-cyclic Silicones | ~40 | ~40 | ~40 | ~40 | ~40 | ~80 | ~80 | ~80 |
| Palmitoylethanolamide | 0-0.5 | 0-0.5 | 0-0.5 | 0-0.5 | 0-0.5 | — | 0.5 | 1 |
| Cannabinoid(s) | 0.1-10 | 0.1-10 | 0.1-10 | 0.1-10 | 0.1-10 | 0.1-10 | 0.1-10 | 0.1-10 |
| Bisabolol | 0.1-1 | 0.1-1 | 0.1-1 | 0.1-1 | 0.1-1 | 0.1-1 | 0.1-1 | 0.1-1 |
| Dimethyl Isosorbide | — | — | — | — | — | 1-10 | 1-10 | 1-10 |
| Caprylic/Capric Triglyceride | — | — | — | — | — | 1-5 | 1-5 | 1-5 |
| PPG-12/SMDI Copolymer | — | — | — | — | — | 1-5 | 1-5 | 1-5 |
| Persa Grattisima Oil | — | — | — | — | — | 1 | 2 | 1-4 |
| Ethoxydiglycol | — | — | — | — | — | 0.5-5 | 0.5-5 | 0.5-5 |
| Oleic Acid | — | — | — | — | — | 0.1-3 | 0.1-3 | 0.1-3 |
| Arena Sativa Kernel Flour | — | — | — | — | — | 0.1-2 | 0.1-2 | 0.1-2 |

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove, and that many modifications and other embodiments are intended to be included within the scope of the appended claims. Moreover, although specific terms are employed herein, as well as in the claims which follow, they are used only in a generic and descriptive sense, and not for the purposes of limiting the described invention, nor the claims which follow.

1-104. (canceled)

105. A personal care composition comprising:
from about 0.1 wt. % to about 10 wt. %, of a cannabinoid;
from about 0.1 wt. % to about 5 wt. % of a PPAR-α agonist;
from about 0.1 wt. % to about 10 wt. % of a terpene; and
from about 80 wt. % to about 95 wt. % of a cosmetically acceptable carrier.

106. The personal care composition according to claim 105, wherein the cannabinoid is selected from: tetrahydrocannabinol; tetrahydrocannabinolic acid; cannabidiol; cannabidiolic acid; cannabinol; cannabigerol; cannabichromene; cannabicyclol; cannabivarin; tetrahydrocannabivarin; cannabidivarin; cannabichromevarin; cannabigerovarin; cannabigerol monomethyl ether; cannabielsoin; cannabicitran; and a combination of two or more thereof.

107. The personal care composition according to claim 105, wherein the terpene is selected from: from 7,8-dihydroionone, Acetanisole, Acetyl Cedrene, Anethole, Anisole, Benzaldehyde, Bergamotene (α-cis-Bergamotene) (α-trans-Bergamotene), Bisabolol (α-Bisabolol), Borneol, Butanoic/Butyric Acid, Cadinene (α-Cadinene) (γ-Cadinene), Cafestol, Caffeic acid, Camphene, Camphor, Capsaicin, Carene (Δ-3-Carene), Carotene, Carvacrol, Carvone, Dextro-Carvone, Laevo-Carvone, Caryophyllene (β-Caryophyllene), Caryophyllene oxide, Castoreum Absolute, Cedrene (α-Cedrene) (β-Cedrene), Cedrene Epoxide (α-Cedrene Epoxide), Cedrol, Cembrene, Chlorogenic Acid, Cinnamaldehyde (α-amyl-Cinnamaldehyde) (α-hexyl-Cinnamaldehyde), Cinnamic Acid, Cinnamyl Alcohol, Citronellal, Citronellol, Cryptone, Curcumene (α-Curcumene) (γ-Curcumene), Decanal, Dehydrovomifoliol, Diallyl Disulfide, Dihydroactinidiolide, Dimethyl Disulfide, Eicosane/Icosane, Elemene (0-Elemene), Estragole, Ethyl acetate, Ethyl Cinnamate, Ethyl maltol, Eucalyptol/1,8-Cineole, Eudesmol (α-Eudesmol) (μ-Eudesmol) (γ-Eudesmol), Eugenol, Euphol, Farnesene, Farnesol, Fenchol (β-Fenchol), Fenchone, Geraniol, Geranyl acetate, Germacrenes, Germacrene B, Guaia-1(10), 11-diene, Guaiacol, Guaiene (α-Guaiene), Gurjunene (α-Gurjunene), Herniarin, Hexanaldehyde, Hexanoic Acid, Humulene (α-Humulene) (13-Humulene), Ionol (3-oxo-α-ionol) (β-Ionol), Ionone (α-Ionone) (β-Ionone), Ipsdienol, Isoamyl acetate, Isoamyl Alcohol, Isoamyl Formate, Isoborneol, Isomyrcenol, Isopulegol, Isovaleric Acid, Isoprene, Kahweol, Lavandulol, a licorice root extract or derivative thereof (e.g. glycyrrhizic acid or glycyrrhetinic acid) Limonene, γ-Linolenic Acid, Linalool, Longifolene, α-Longipinene, Lycopene, Menthol, Methyl butyrate, 3-Mercapto-2-Methylpentanal, Mercaptan/Thiols, 0-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (β-Myrcene), γ-Muurolene, Nepetalactone, Nerol, Nerolidol, Neryl acetate, Nonanaldehyde, Nonanoic Acid, Ocimene, Octanal, Octanoic Acid, P-cymene, Pentyl butyrate, Phellandrene, Phenylacetaldehyde, Phenylethanethiol, Phenylacetic Acid, Phytol, Pinene, β-Pinene, Propanethiol, Pristimerin, Pulegone, Retinol, Rutin, Sabinene, Sabinene Hydrate, cis-Sabinene Hydrate, trans-Sabinene Hydrate, Safranal, α-Selinene, α-Sinensal, β-Sinensal, β-Sitosterol, Squalene, Taxadiene, Terpin hydrate, Terpineol, Terpine-4-ol, α-Terpinene, γ-Terpinene, Terpinolene, Thiophenol, Thuj one, Thymol, α-Tocopherol, Tonka Undecanone, Undecanal, Valeraldehyde/Pentanal, Verdoxan, α-Ylangene, Umbelliferone, Vanillin; and a combination of two or more thereof.

108. The personal care composition according to claim 105, further comprising a flavonoid selected from: quercetin; rutin; quercitrin; catechin; gallocatechin; catechin 3-gallate; gallocatechin 3-gallate; epicatechin; epigallocatechin; epicatechin 3-gallate; epigallocatechin 3-gallate; theaflavin-3-gallate; theaflavin-3′-gallate; theaflavin-3,3′-digallate; kaempferol; myricetin; fisetin; galangin; isorhamnetin; pachypodol; rhamnazin; a pyranoflavonol; furanoflavonol; luteolin; resveratrol; apigenin; tangeritin; hesperetin; naringenin; eriodictyol; homoeriodictyol; and a combination of two or more thereof.

109. The personal care composition according to claim 105, wherein the cosmetically acceptable carrier comprises a hydrophobic component comprising an oil comprising greater than about 50% polyunsaturated fatty acids.

110. The personal care composition according to claim 109, wherein the oil is selected from corn oil; cottonseed oil; flaxseed/linseed oil; hemp/hempseed oil; soybean oil; and a combination of two or more thereof.

111. The personal care composition according to claim 109, wherein the hydrophobic component further comprises a silicone component.

112. The personal care composition according to claim 111, wherein the silicone component comprises polydimethylsiloxane and silicone crosspolymer.

113. The personal care composition according to claim 112, wherein the polydimethylsiloxane and silicone crosspolymer are present in a weight ratio of about 4:1.

114. The personal care composition according to claim 105, wherein the composition is substantially free of cyclic silicones.

115. A personal care composition comprising:
a cannabinoid;
a terpene;
Avena sativa (oat) kernel flour; and
a cosmetically acceptable carrier.

116. The personal care composition according to claim 115, further comprising a PPAR-α agonist.

117. The personal care composition according to claim 116, wherein the PPAR-α agonist comprises a fatty acid amide.

118. The personal care composition according to claim 117, wherein the fatty acid amide comprises palmitoylethanolamide.

119. The personal care composition according to claim 115, further comprising a penetration enhancing system comprising a compound selected from glyceryl oleate, ethanol, dimethyl sulfoxide, N-lauroyl sarcosine, ethoxydiglycol, dimethyl isosorbide, sodium lauryl sulfoacetate, isopropyl myristate, methyl laurate, propylene glycol, oleic acid, sodium octyl sulfate, eucalyptol, and a combination of two or more thereof.

120. A personal care composition comprising:
petrolatum or a wax of similar consistency;
a microcrystalline wax;
a cannabinoid;
a terpene; and
a cosmetically acceptable carrier.

121. The personal care composition according to claim 120, wherein the petrolatum is present in an amount greater than the amount of microcrystalline wax.

122. The personal care composition according to claim 121, wherein the composition further comprises from about 0.1 wt. % to about 10 wt. % menthol.

123. A method of treating, preventing or ameliorating a symptom associated with a disease, disorder, or condition of the skin, comprising applying a personal care composition according to claim 105, to the skin of a subject in need thereof; wherein the disease, disorder, or condition of the skin is selected from: acne, dermatitis (e.g. atopic dermatitis, contact dermatitis, seborrheic dermatitis, or stasis dermatitis), eczema (e.g. dyshidrotic eczema, hand eczema or nummular eczema), itch, neruodermatitis, lichen simplex chronicus, dry skin, baby rash, psoriasis, pruritus (e.g. neuropathic pruritic), burns, itch associated with burns, flaky skin, inflammation, keloids, epidermolysis bullosa.

124. A method of treating or preventing, or ameliorating a symptom associated with pain comprising: applying a personal care composition according to claim 105, to the skin of a subject in need thereof; wherein the pain is associated with a disease, disorder or condition selected from: arthritis (e.g. osteoarthritis, psoriatic arthritis or rheumatoid arthritis); bursitis; carpal tunnel syndrome; central pain syndrome; cervical stenosis; complex regional pain syndrome; degenerative disc disease; peripheral neuropathy (e.g. diabetic peripherial neuropathy); Ehlers-Danlos syndrome; failed back surgery syndrome; fibromyalgia; headache; a herniated disc; a knee injury; migraine; myositis; pinched nerve; sciatica; spinal cord injury; spinal stenosis; TMJ, trigeminal neuralgia; whiplash; post herniorraphy pain syndrome; post mastectomy pain syndrome; post thorocotomy pain syndrome; trigger points; lupus; lyme disease; ulcerative colitis; scoliosis; spasmodic torticollis; Scheuemann's kyphosis disease; sacroiliac joint dysfunction; radiculopathy; postherpetic neuralgia (shingles); polymyositis; multiple sclerosis; juvenile dermatomyositis; irritable bowel sydrome; intercostal neuralgia; Guillain Barre syndrome; gout; glossopharyngeal neuralgia; eosinophilia-myalgia syndrome; endometriosis; dystonia; dermatomyositis; Crohn's disease; Ankylosing Spondylitis; Arachnoiditis; Arthrofibrosis; Avascular Necrosis; Burning Mouth Syndrome; Cadasil; Cauda Equina Syndrome; Charcot-Marie-Tooth (CMT) Disease; Chronic Fatigue Syndrome; cerebral palsy; AIDS-related pain; back pain; cancer pain; growing pains; knee pain; leg pain; myofascial pain; neck pain; muscular pain; exercised induced inflammatory pain; neuropathic pain; pelvic pain; phantom limb pain; post stroke pain; and vascular pain.
